(19) United States
(12) Patent Application Publication
(10) Pub. No.: US 2004/0142394 A1
(43) Pub. Date: Jul. 22, 2004

(54) Method for diagnosing a tumor in a patient determining the concentration of PIBF

(76) Inventors: Julia Szekeres-Bartho, Pecs (HU); Eszter Nagy, Vienna (AT); Beata Polgar, Pecs (HU); Tamas Palkovics, Keszthely (HU); Margit Koch, Vienna (AT); Christoph Klade, Neustadt (AT); Tamas Henics, Vienna (AT)

Correspondence Address: BAKER & BOTTS, 30 ROCKEFELLER PLAZA, NEW YORK, NY, 10112

(21) Appl. No.: 10/444,693

(22) Filed: May 23, 2003

Related U.S. Application Data
(63) Continuation of application No. PCT/EP01/13876, filed on Nov. 28, 2001.

(30) Foreign Application Priority Data
Nov. 28, 2000 (AT) A 1997/2000

(51) Int. Cl. G01N 33/574

(52) U.S. Cl. 435/7.23

(57) ABSTRACT

The present invention relates to a method for diagnosing a tumor in a patient said method comprising measuring the concentration of Progesterone-Induced Immunomodulatory Protein (PIBF) or a derivative thereof or a fragment thereof in the sample taken from the patient, and to the use of an anti-PIBF or a derivative thereof or a fragment thereof, and a polynucleotide endoding PIBF, respectively, as an anti-tumor medicine.

The present invention concerns a recombinant protein with a Progesterone-Induced Immunomodulatory Protein-(PIBF-)activity, a nucleic acid molecule encoding a recombinant protein with a PIBF activity, a nucleic acid vector comprising said nucleic acid sequence, a cell comprising said vector and a method for diagnosing a tumor in a patient.

For the maintenance of normal pregnancy, the production of progesterone—a steroid hormone with wide range of immunosuppressive effects—is absolutely essential. Peripheral lymphocytes from healthy pregnant women express nuclear receptors to sense this hormone (Szekeres-Bartho et al. J.Reprod.Immunol. 16, 239 (1989); Szekeres-Bartho et al. Cell. Immunol. 125, 273 (1990)), and produce a mediator protein named the Progesterone Induced Blocking Factor (PIBF) (Szekeres-Bartho et al. Am.J.Reprod.Immunol. Microbiol. 9, 15 (1985)). The sequence of the PIBF cDNA from human liver showed no significant homology to that of any of the known proteins (HSPIBF, Acc. No. Y09631). The encoded precursor protein is highly hydrophilic and has a molecular weight of 89 kDa. The naturally occuring PIBF as discoverred originally is a 34-36 kDa immunomodulatory protein with a sequence length of 757 amino acids.

It has been determined that the concentration of PIBF in urine samples of healthy persons is about 1-10 ng/ml whereas the concentration of PIBF in pregnant women from the 2^{nd} trimester ranges from about 70-150 ng/ml. These high levels quickly return to normal following abortion or labor.

PIBF which mediates the effects of progesterone is shown to have very potent immune modulatory function, both in vitro, as well as in vivo. Indeed, PIBF is shown to be essential for pregnancy in a mouse model, since PIBF isolated from culture supernatants of mouse lymphocytes protects fetuses from resorption induced by an-tiprogesterones. In addition, neutralizing antibodies against the mouse PIBF cause resorption of embryos and consequently abortus. The important role of PIBF in human reproduction has also been substantiated by measuring low levels in body fluids from pathologic pregnancies. PIBF plays an important role in the maintenance of pregnancy most likely by inhibiting natural killer lymphocytes. Importantly, by experimentally manipulating the amount of PIBF in vitro, one can modulate the killing activity of peripheral blood lymphocytes containing NK (natural killer) cells. It has been determined that there are at least two mechanisms of action of PIBF on NK cells. One is a direct inhibition of NK cell activity. NK cells kill their target cells by exocytosis of perforin and serine esterase-containing granules in the contact area between effector and target cells. Decidual lymphocytes—of which 60% carry NK surface markers—have high perforin content, however, they exert low cytotoxic activity. Although activated NK cells find and bind their targets in the presence of PIBF, they fail to release perforin from the storage granules, and as a result, there is no lysis of target cells. It seems that PIBF paralyses NK cells and holds the cytotoxic machinery under restraint by inhibiting degranulation and thus the release of killing substances.

There is another, indirect mechanism, by which PIBF exerts its anti-NK effect, through altered cytokine expression. In the presence of PIBF there is a significant decrease in TNFα (Tumor Necrosis Factor α) production by NK cells, which might also be involved in the down-regulation of NK activity. The amount of secreted TNFα is inversely related to PIBF production both in vitro and in vivo.

The second main mechanism of action of PIBF is the induction of T_{H2} cytokine dominance. T_{H2} dominance contributes to decreased cell mediated responses and B cell enhancement, whereas T_{H1} dominance results in decreased humoral responses and favors cellular immunological mechanisms. The secreted PIBF facilitates the production of T_{H2} cytokines, such as IL-3, IL-4 and IL-10, while it suppresses T_{H1} cytokines, such as IL-12 and IFN-γ both in vitro and in vivo. Neutralization of PIBF by specific antibodies results in a T_{H1} shift in vivo, which is also a characteristics of failed pregnancies. The effect of PIBF on humoral immune responses is not only a simple enhancement but induction of the production of asymmetric antibodies. This is a population of antibodies (ab) which, owing to the presence of a mannose-rich oligosaccharide residue on one of the Fab arms of the molecule, possesses an asymmetric structure, and has no or low effector functions; however, these ab might act as blocking antibodies. The ratio of asymmetric IgG was significantly higher in supernatants of hybridoma cells cultured in the presence of PIBF than in those cultured in the absence of PIBF. Further studies revealed a positive relationship between asymmetric antibody content of the sera and PIBF expression on lymphocytes. Furthermore, blocking of progesterone receptors by RU 486 or neutralizing endogenous PIBF activity by specific anti-PIBF antibodies significantly reduced the production of asymmetric antibodies in pregnant mice.

Malignant tumors, i.e., cancers, are the second leading cause of death in all developed countries after heart disease and develop in one in three persons. One of every four persons dies of cancer. Cancer is characterized primarily by an increase in the number of abnormal, or neoplastic, cells derived from a normal tissue which proliferate to form a tumor mass, the invasion of adjacent tissues by these neoplastic tumor cells, and the generation of malignant cells which spread via the blood or lymphatic system to regional lymph nodes and to distant sites. The latter progression to malignancy is referred to as metastasis.

Cancer can result from a breakdown in the communication between neoplastic cells and their environment, including their normal neighbouring cells. Signals, both growth-stimulatory and growth-inhibitory, are routinely exchanged between cells within a tissue. Normally, cells do not divide in the absence of stimulatory signals, and, likewise, will cease dividing in the presence of inhibitory signals. In a cancerous, or neoplastic, state, a cell acquires the ability to “override” these signals and to proliferate under conditions in which normal cells would not grow.

Tumor cells must acquire a number of distinct aberrant traits to proliferate. Reflecting this requirement is the fact that the genomes of certain well-studied tumors carry several different independently altered genes, including activated oncogenes and inactivated tumor suppressor genes. Each of these genetic changes appears to be responsible for imparting some of the traits that, in aggregate, represent the full neoplastic phenotype.

Tumor cells carry antigens that can be recognized as being foreign to the body and that is one of the major functions of the immune system to eliminate such cells before they can form large tumors. This immune surveillance is clearly ineffective in patients with progressive malignant diseases. There is a range of protective measures identified that suppresses self-reactivity and may represent a major barrier in the immune system's ability to eradicate tumor cells. There are a number of mechanisms exerted by tumor cells such as 1. non-expression of classical and expression of non-classical self-identifying class I MHC molecules (such as HLA-G), which undermines the killing effect of (tumor)antigen specific class I MHC-restricted CTLs; 2. biasing towards T_{H2} responses, suppressing T_{H1} helper function, and consequently effective cytotoxic anti-tumor responses; and 3. the production of immunosuppressive factors that down-regulate local and systemic immune responses (for example, secretion of TGF-β decreases T cell proliferation and cytotoxicity, expression of fas ligand, which induces apoptosis of CTLs.). As a result of these cumulative effects, tumors enjoy an immunologically privileged state, and grow without or with restricted control of the immune system.

It is fairly well established that many pathological conditions, such as infections, cancer, autoimmune disorders, etc., are characterized by the inappropriate expression of certain molecules. These molecules thus serve as “markers” for a particular pathological or abnormal condition. Apart from their use as diagnostic “targets”, i.e., materials to be identified to diagnose these abnormal conditions, the molecules serve as reagents which can be used to generate diagnostic and/or therapeutic agents.

An aim of the present invention is a novel method for diagnosing a tumor in a patient which can be carried out easily and safely, which method does not require high-tech equipment, does not cause any particular trouble to the patient, which can be carried out quickly and which leads to results which allow to distinguish between a patient with a tumor and a healthy patient.

A further object of the present invention is to provide a kit for carrying out the method for diagnosing a tumor in a patient.

A still further object of the present invention is to provide an efficient anti-tumor medicine.

The method according to the present invention for diagnosing a tumor in a patient with which the above object is solved comprises taking a sample from the patient, measuring the concentration of PIBF (Progesterone Induced Blocking Factor) or a derivative thereof or a fragment thereof in the sample and determining whether the concentration of PIBF in the sample is above or below a predetermined threshold value, whereby the concentration above the threshold value identifies a patient with a tumor.

During the characterization of PIBF as an important immunomodulatory molecule for the maintenance of pregnancy, it has surprisingly been shown that tumor cells express PIBF or PIBF related substances, whereas no or low PIBF reactivity is found in the adjacent normal tissues. This indicates that PIBF is involved in the development or maintenance of immunological tolerance towards malignantly transformed cells and constitutes therefore a useful marker for tumor cells.

Therefore the method according to the present invention uses the fact that the concentration of PIBF in a sample taken from the patient to be tested is higher than the concentration of PIBF in a sample taken from a healthy person.

In the scope of the present invention, the sample taken from the patient may be any kind of sample, fluid or not, from practically any part of the body. The concentration of the PIBF can be measured according to any method known in the art which enables to quantify the concentration of PIBF in a sample. This may comprise chemical, microbiologic, physical techniques, staining, etc. on fluids, tissue samples, etc. Possible methods include in vivo imaging with Computer Tomograph (CT) and Magnetic Resonance Image (MRI), after labeling with radionucleic and paramagnetic (e.g. gadolinium) labels, respectively, etc.

Since the PIBF may be submitted to metabolic or other changes in the body of the patient, the PIBF may comprise modifications depending on which sample has been taken from the patient. The PIBF may for example have been cleaved so that only a fragment of the PIBF is present in the given sample. The PIBF may furthermore have been modified so that a derivative of the PIBF is present in that sample or also a fragment of that derivative. It has also been shown that alternatively processed PIBF mRNA are present in tumor cells in a different concentration compared to normal cells, therefore, proteins or fragments translated from these different forms of mRNA molecules or fragments thereof are also comprised by the the term “fragments”. Therefore, also the PIBF derivative, or a fragment of the PIBF or of the PIBF derivative or PIBF related substances (such as e.g. a cleaved product of 34 kDa or an alternatively spliced 14 kDa product) can be used as an indication of the concentration of the PIBF in the patient and therefore the respective concentration can be used for the method for diagnosing a tumor in a patient according to the present invention.

In the scope of the present invention the term “PIBF or fragments thereof” refers to—without being limited—to sequences according to SEQ ID Nos: 1, 3, 4, 6, 8, 10, 14, 15, 17, 19, 20, 23, 25, 27, 29, 31, 32, 34 and 36 or fragments or derivatives thereof. Therefore, examples of PIBF or fragments thereof which can be detected or quantified according to the present invention are these above mentioned sequences. Since it has been shown that exons 17 and 18 are included in almost all mRNA forms which have been identified PIBF fragments comprising exons 17 and 18 (see figs.) are preferably used for the detection or quantification of PIBF in a sample.

In the scope of the present invention, the fragment of the PIBF or of the PIBF-derivative may comprise for example less than 715 amino acids, preferably less than 500 amino acids, still preferred less than 200 amino acids, and most preferred less than 50 amino acids.

In the scope of the present invention, the term “derivative” comprises for example any naturally or even non naturally occurring modifications, e.g. cleavage, glycosylations, methylations, acetylations, amidations, phosphorylations, sulfatations, deletions, substitutions, etc.

Also, in the scope of the present invention, “threshold value” relates to a concentration value which will generally be the median sample concentration of PIBF in healthy sample donors. It is possible to take a known general median PIBF concentration in healthy people according to the literature or also to determine the sample concentration of PIBF in healthy donors when carrying out the present invention. The threshold value may also be determined in healthy (normal) samples taken earlier (in a healthy state) from the same person. Examples of such threshold values may be for example between 1 and 10 ng/ml, preferably between 1 and 5 ng/ml, whereby the concentration depends on the method of detection as well as the type of tumor. Furthermore, the threshold value may be zero in the case that alternatively processed PIBF mRNA products are present only in tumor cells and not in healthy cells. Therefore, the threshold value also depends on the PIBF molecule and must be determined for each specific PIBF molecule individually.

However, when determining the threshold value it is important that the sample from the healthy person is not taken from a pregnant woman since the PIBF concentration in samples of pregnant women is higher than the PIBF concentration in samples of non-pregnant women.

The concentration of PIBF measured in the sample taken from the patient which is above the predetermined threshold value identifies individuals with suspected tumor. A “tumor” as used herein refers to all neoplastic cell growth and proliferation, whether malignant or benign, and all pre-cancerous and cancerous cells and tissues.

Within the term “patient”, in the scope of the present invention, patients with a tumor but also patients susceptible of having a tumor as well as healthy people who are having a general routine check are comprised. Of course the term “patient” may comprise also any animal, in particular mouse, rat, guinea pig, monkey, which animal is preferably a laboratory animal used for analyses, e.g. for detecting specific tumors, testing anti-tumor substances or carcinogenic substances. Furthermore, the animal may be a genetically modified animal which has a pre-disposition for developing tumors.

Since pregnancy also results in elevated PIBF levels, sexually active women have to be tested by conventional pregnancy tests (e.g. based on hCG) before considered as patients with tumor. It also means that it is very difficult to use this test to detect tumor growth when the patient is a pregnant woman beyond the first trimester. However, since a significant portion of the pregnancy-related malignancies are related to the uncontrolled growth of pregnancy-related tissues (such as trophoblast cells in mola hydatidosa), extremely high levels (>150-200 ng/ml) of PIBF might indicate tumor growth with or without a viable baby present.

Preferably, the tumor to be diagnosed by the method according to the present invention is an epithelial carcinoma. Since the vast majority of human tumors (based on world wide morbidity data) are epithelial carcinomas (lung, breast, colon, etc.) the method according to the present invention is particularly advantageous for the diagnosis of this kind of tumor.

The epithelial carcinoma is preferably a lung carcinoma, colon carcinoma and breast carcinoma, respectively. The PIBF concentration in samples taken from patients with the above mentioned tumors is particularly high compared to the PIBF concentration in samples from healthy patients. Therefore, if the method according to the present invention is used for diagnosing one of the above mentioned tumors a concentration above a threshold value identifies individuals with a suspected tumor. However, a concentration below the threshold value does not necessarily exclude the presence of a tumor in certain cases.

According to an advantageous embodiment of the present invention the sample is a body fluid, preferably urine and serum, respectively. This enables a very simple way of taking the sample from the patient without any surgical step and without the necessity of specific high-tech instruments. The body fluid can be taken in any laboratory or even at the patient's home and is especially advantageous for a routine diagnosis, a diagnosis on a patient who is very weak and for regular checking of the progression of the tumor in a patient. The PIBF concentration can for example be measured with a dry chemistry method, e.g. a strip which will change its colour according to the PIBF concentration in a sample into which it is dipped.

Alternatively, the sample is a tissue sample. Even though the taking of this kind of sample from the patient is not as easy as the taking of a body fluid a method according to the present invention using a tissue sample from the patient enables the direct location of the tumor, especially if different tissue samples are taken and compared with each other. Furthermore, it is possible to directly follow the progressing of the tumor. Furthermore, by detecting the tissues with a tumor the method according to the present invention can further be used at least as an additional method for deciding which tissues and which parts of a body of the patient must be surgically removed.

According to preferred embodiment of the present invention the threshold value is the concentration of PIBF in a sample of a healthy person. Of course, the threshold value is particularly precise if it is the median concentration of PIBF of a plurality of samples of healthy persons.

Preferably, the threshold value is determined by measuring the concentration of PIBF in a sample of at least one healthy person parallel to the determination of the concentration of PIBF in a sample of the patient. Since the measured concentration depends on the method of measuring the PIBF concentration, the diagnosis is more specific and exact if the method for measuring the concentration of PIBF in the sample of the patient and in the sample of the healthy person are identical. In order to further enhance the sensitivity of the method the sample from the patient and the sample from the healthy person are preferably measured in parallel, e.g. at the same time in order to eliminate any interfering parameters, e.g. temperature, buffers etc. which have an influence on the result. The sample of the healthy person will preferably be measured in parallel as the “negative sample”.

Advantageously, as a positive control the concentration of PIBF or a derivative thereof or a fragment thereof in a sample comprising a defined concentration of PIBF or a derivative thereof or a fragment thereof is measured parallel to the determination of the concentration of PIBF in the sample of the patient. The measuring in parallel of the positive control allows to control the results and detect any divergence in the method.

Preferably, the concentration of PIBF in the sample is measured immunologically, in particlular by a competitive assay, by a sandwich assay, by immunostaining or combinations of these methods. Any immunological method known to the person skilled in the art may be applied. Immunological methods are highly sensitive methods for detecting molecules and therefore particularly advantageous for the measuring of the PIBF concentration in the sample. To carry out the immunological method it is necessary to have at least one anti-PIBF antibody which will specifically bind to PIBF, derivatives thereof or fragments thereof. The antibody may be monoclonal or polyclonal and may further be recombinant. Furthermore, humanized monoclonal or phage encoded single-chain monoclonal antibodies may be used.

Examples of recombinant monoclonal anti-human PIBF antibodies which can be used as described above are deposited at the hybridoma cell bank at the University Medical School of Pécs, Department of Immunology and Biotechnology, Hungary, under the deposition Nos. 11 to 14/2001, cell line codes HYB 255-258.

“Single-chain antibodies” are structurally defined as comprising the binding portion of a first polypeptide from the variable region of an antibody, associated with the binding portion of a second polypeptide from the variable region of an antibody, the two polypeptides being joined by a peptide linker linking the first and second polypeptides into a single polypeptide chain. The single polypeptide chain thus comprises a pair of variable regions connected by a polypeptide linker. The regions may associate to form a functional antigen-binding site, as in the case wherein the regions comprise a light-chain and a heavy-chain variable region pair with appropriately paired complementarity determining regions (CDRs).

The term “humanized antibody” as used herein refers to antibody molecules in which amino acids have been replaced in the known antigen binding reagents in order to more closely resemble a human antibody and still retaining the original binding ability.

The antibodies may be generated using methods that are well known in the art. Such antibodies may include but are not limited to polyclonal, monoclonal, recombinant, chimeric, single chain, Fab fragments, and fragments produced by a Fab expression library. Neutralizing antibodies, (i.e., those which inhibit dimer formation) are especially preferred for therapeutic use.

For the production of antibodies, various hosts including goats, rabbits, rats, mice, chicken (Yab), humans, and others, may be immunized by injection with PIBF natural or recombinant protein or any fragment or oligopeptide thereof which has immunogenic properties or a PIBF-DNA (fragment). Depending on the host species, various adjuvants may be used to increase immunological response. Such adjuvants include but are not limited to Freund's, mineral gels such as aluminium hydroxide, and surface active substances such as lysolecithin, pluronic polyols, polyanions, aluminium, polycations (e.g. polyArg), peptides, oil emulsions, keyhole limpet hemocyanin, and dinitrophenol. Among adjuvants used in humans, BCG (bacilli Calmette-Guerin) and Corynebacterium parvum are especially preferable.

It is preferred that the peptides, fragments, or oligopeptides used to induce antibodies to PIBF have an amino acid sequence consisting of at least five amino acids, and more preferably at least 10 amino acids. It is also preferable that they are identical to a portion of the amino acid sequence of the natural protein. Short stretches of PIBF amino acids may be fused with those of another protein such as keyhole limpet hemocyanin and antibodies will be produced against the chimeric molecule.

Monoclonal antibodies to PIBF may be prepared using any technique which provides for the production of antibody molecules by continuous cell lines in culture. These include but are not limited to the hybridoma technique, the human B-cell hybridoma technique, and the EBV-hybridoma technique.

In addition, techniques developed for the production of “chimeric antibodies”, the splicing of mouse antibody genes to human antibody genes to obtain a molecule with appropriate antigen specificity and biological activity can be used. Alternatively, techniques described for the production of single chain antibodies may be adapted, using methods known in the art, to produce PIBF-specific single chain antibodies. Antibodies with related specificity, but of distinct idiotypic composition, may be generated by chain shuffling from random combinatorial immunoglobulin libraries.

Antibodies may also be produced by inducing in vivo production in the lymphocyte population or by screening recombinant immunoglobulin libraries or panels of highly specific binding reagents.

Antibody fragments which contain specific binding sites for PIBF may also be generated. For example, such fragments include but are not limited to the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity.

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between PIBF and its specific antibody. A two-site, monoclonal-based immunoassay utilizing monoclonal antibodies reactive to two non-interfering PIBF epitopes is preferred, but a competitive binding assay may also be employed.

In one preferred form the concentration of PIBF in the sample is measured by a competitive assay. According to this method a solid phase is covered with preferably recombinant human PIBF (or its variants) at a specific concentration. Labelled anti-PIBF antibodies are added together with the examples to be measured. The higher is the PIBF concentration in the sample the lower is the corresponding detected value. Based on these readings the absolute concentration of the PIBF can be determined. This is a particularly precise method especially when the sample is a body fluid and can be carried out for example with an ELISA.

According to a further preferred embodiment of the invention, the concentration of PIBF in a sample is measured by a sandwich assay. For this assay it is necessary to have two anti-PIBF antibodies which each bind to a different epitope of the PIBF molecule. The first anti-PIBF antibody is preferably immobilized to a solid support after which the sample which is to be measured is added so that the PIBF present in the sample binds to the first anti-PIBF antibody. A second anti-PIBF antibody which is preferably labelled is added so that it binds to the bound PIBF. The amount of bound second anti-PIBF antibody is measured and is used as indication for the absolute concentration of the PIBF in the sample. Also this method is preferably used when the sample to be measured is a body fluid of the patient and can be carried out by ELISA.

According to another preferred embodiment to the present invention the concentration of PIBF in a sample is measured by immunostaining. This method is preferably used when the sample which is to be measured is a tissue sample of the patient. According to this method anti-PIBF antibody is directly added to the tissue sample of the patient where it binds to PIBF present in the tissue sample. The bound antibody is quantified there by directly indicating the concentration of PIBF in the tissue sample. This method allows localisation of PIBF in a sample.

Preferably the concentration of PIBF is measured indirectly by measuring the concentration of PIBF-mRNA in the sample. For this polynucleotides may be used, including oligonucleotide sequences, antisense RNA and DNA molecules, and PNAs. The polynucleotides may be used to detect and quantitate gene expression in samples in which expression of PIBF will be correlated with a tumor. Accordingly, a kit can be provided comprising a reagent comprising the above mentioned (labelled) polynucleotides in order to carry out PIBF-mRNA measurement in the given sample. Here again it is further preferable to measure the concentration of alternatively processed mRNA. Also, the presence or absence of a specific mRNA molecule can give information with respect to whether or not the cells are tumor cells.

In one aspect, hybridization with nucleotide probes may be used to identify PIBF-mRNA sequences. Nucleotide sequences complementary to the PIBF-mRNA may be labelled by standard methods, and added to a fluid or tissue sample from a patient under conditions suitable for the formation of hybridization complexes. After a suitable incubation period, the sample is washed and the signal is quantitated and compared with the threshold value.

The specificity of the probe, whether it is made from a highly specific region or a less specific region and the stringency of the hybridization (maximal, high, intermediate, or low) will determine whether the probe identifies only naturally occurring sequences encoding PIBF, alleles, or related sequences.

Probes which are used for the hybridization of PIBF-mRNA (related) sequences should preferably show at least 50%, preferably 70%, still preferred 90%, homology to the PIBF encoding sequence or fragments thereof. The hybridization probes of the subject invention may be DNA or RNA and derived from the nucleotide sequence of SEQ ID. NO 3 or 5 (PIBF-cDNA).

Examples of such PIBF-mRNA molecules to be detected and/or quantified are for example those detected by DNA or RNA derived from the nucleotide sequence of SEQ ID NO: 5, 7, 9, 11, 12, 13, 16, 18, 21, 22, 24, 26, 28, 30, 33, 35 and 37. Since it has been shown that exons 17 and 18 are included in almost all mRNA forms which have been identified DNA or RNA derived from a sequence coding exons 17 and 18 (see figs.) are preferably used for the detection or quantification of PIBF-mRNA in a sample.

Hybridization probes may be labelled by a variety of reporter groups, for example, radionucleotides such as 32P or 35S, or enzymatic labels, such as alkaline phosphatase coupled to the probe via avidin/biotin coupling systems, and the like.

The polynucleotide sequences encoding PIBF may further be used in northern blot analysis, dot blot, or other membrane-based technologies; in dip stick, pin, ELISA or (micro-) chip assays utilizing fluids or tissues from patient biopsies to detect PIBF-mRNAs. Such methods are well known in the art.

Additionally, PIBF-mRNA can be detected and measured by RT-PCR: In a first step the mRNA is transcribed by reverse transcriptase into cDNA after which the cDNA is detected and quantified by PCR. The oligomers for the PCR may be chemically synthesized, generated enzymatically, or produced from a recombinant source. Oligomers will preferably consist of two nucleotide sequences, one with sense orientation and another with antisense, employed under optimized conditions for identification of the specific sequence. The same two oligomers, nested sets of oligomers, or even a degenerate pool of oligomers may be employed under less stringent conditions for detection and/or quantitation of closely related sequences.

A further aspect of the present invention relates to a method for determining the positive or negative progression of a tumor in a patient comprising diagnosing a tumor in a patient according to one of the above mentioned methods according to the present invention and determining whether the measured concentration of PIBF or a derivative thereof or a fragment thereof in the sample is above or below at least one previously measured concentration of PIBF or a derivative thereof or a fragment thereof in at least one sample previously taken from the same patient, whereby the concentration above the previously measured concentration identifies a positive progression. Since the concentration of PIBF in a sample is directly proportional to the progression of the tumor, e.g. size, development etc., the method according to the present invention allows direct analysing of the course of the illness. For a complete characterisation of the progression of the tumor it is of course advantageous to take many samples over a period of time in particular before and after a specific treatment, e.g. with a substance or by removing tumor tissue completely or partly, in which case the effectiveness of the specific treatment can be analysed. The term “positive progression” used herein means that the tumor is further developing.

A further aspect of the present invention relates to the use of an anti-PIBF antibody or a fragment thereof in an above described method according to the present invention. As mentioned above the anti-PIBF antibody may be monoclonal, polyclonal, it further may be recombinant, humanized or phage encoded single-chain antibody. If only a fragment of the antibody is used, this fragment comprises the epitope of the anti-PIBF antibody which recognizes the PIBF.

It is preferred to use a monoclonal antibody in order to achieve a most specific and precise result. The monoclonal antibody may be produced as mentioned above, and the examples given above also apply here.

A further aspect of the present invention relates to the use of PIBF or a derivative thereof or a fragment thereof in one of the above mentioned methods according to the present invention. As already mentioned above the fragment may be a fragment of the PIBF or a fragment of the PIBF derivative. Here, the same definitions and preferred embodiments or examples as mentioned above apply.

Preferably the PIBF is recombinant, meaning that also the derivative or the fragment may be recombinant.

A further aspect of the present invention relates to a kit comprising a first reagent comprising at least one anti-PIBF antibody or a fragment thereof and a second reagent comprising PIBF or a derivative thereof or a fragment thereof at a defined concentration. Of course, the anti-PIBF antibody and the PIBF are present in a form which allows their storage, e.g. in dry, lyophilized, frozen or dissolved form. Further, the kit may comprise any further buffers, enzymes, salts etc., which are necessary for the above mentioned method to be carried out.

Preferably the kit comprises a solid phase to which the at least one anti-PIBF antibody or the fragment thereof or the PIBF or the derivative thereof or the fragment thereof is bound. The solid phase may be any solid phase known to the person skilled in the art, e.g. any insoluble material which can provide a substrate upon which to immobilize proteins or peptides, for example in the form of a dry strip. Such substrates may include nylon, amino acids, glass, cellulose and the like. This kit may preferably be used for competitive or a sandwich assay whereby the further reagent comprising either the antibody or the PIBF, depending on which is immobilized to the solid phase, and the sample are added to the solid phase.

Preferably the PIBF present in the above mentioned kit is recombinant meaning of course that also the derivative thereof and the fragment thereof, respectively, are recombinant.

A preferred kit comprises a further reagent comprising a second anti-PIBF antibody or a fragment thereof which binds to an epitope of the PIBF which is distinct to the epitope recognized by the first anti-PIBF antibody or the fragment thereof. This kit is particularly advantageous in order to carry out a sandwich assay.

The above mentioned kit according to the present invention is preferably for diagnosing a tumor in a patient and for determining the progression of a tumor in a patient, respectively. The methods are the same as described above, whereby the reagent comprising the PIBF or a derivative thereof or a fragment thereof at the defined concentration is used either as a positive control as described above or to carry out a competitive assay as described above (whereby it is used in competition to the PIBF present in the sample of the patient) or both.

A further aspect of the present invention concerns the use of an anti-PIBF antibody or a fragment thereof for the preparation of an anti-tumor medicine. The anti-PIBF antibody or the fragment thereof specifically blocks or neutralizes PIBF, thereby specifically abolishing PIBF activity in tumors thus rendering the tumors susceptible to NK (and potentially CD8+ and other T cell mediated lysis). Furthermore, mono and bi-specific antibodies can. specifically recognize PIBF on a surface of tumor cells and can be used to deliver toxic substances to the tumorous compartment of the body of the patient. The main strategy of the anti-tumor medicine is the use of the knowledge that tumor cells produce higher concentrations of PIBF. With this information which is the basis of the present invention various strategies can be developed for fighting a tumor in a patient.

According to a preferred embodiment, the antibody is a monoclonal, humanized, and single chain antibody, respectively. The above mentioned deposited antibodies may also be used for this aspect of the present invention.

Preferably the antibody has attached thereto a molecule. In this case the anti-PIBF antibody is used as a targeting or delivery mechanism for bringing a molecule, e.g. a pharmaceutical agent, to cells or tissues which express PIBF. The antibody which is administered to the patient binds to the tumor expressing PIBF and thereby brings the molecule which is toxic for the tumor into direct contact with the tumor. There are various methods and molecules which are used which are known to the person skilled in the art. For example a toxic molecule can be used which will enter the tumorous cells and interfere with for example essential metabolic steps thereby killing the cells. Also, the toxic molecule may induce cell lysis or act as a receptor for other toxic substances or enzymes which will kill the tumorous cells. However, independently of the way the toxic molecule functions, the main point is that the molecule is specifically directed to the tumorous cells by the anti-PIBF antibody and does not interfere with healthy cells.

The molecule may preferably be a toxic substance and a prodrug, respectively, in particular a radionuclide, a toxin and a chemotherapeutic drug, respectively. By delivering the substance to the tumorous target an effective anti-tumor medicine is achieved.

A further aspect of the present invention concerns the use of PIBF or a derivative thereof or a fragment thereof for the preparation of an anti-tumor medicine. There are two strategies for these anti-tumor medicines according to the present invention:

A PIBF-derivative or a fragment thereof is used as inhibitory protein or peptide which interferes with the PIBF action through binding and thereby blocking or inactivating putative receptors for PIBF present on cells, e.g. NK cells, or inhibit signalling components down-stream of receptor binding.

According to a preferred embodiment of a present invention the medicine is a vaccine. The PIBF derivative or the fragment thereof comprises the immunogenic peptide of PIBF and can be used for vaccination either to induce antigen specific anti-tumor cytotoxic T cell responses and/or to stimulate the production of neutralizing antibodies by the immune system of the cancer patient himself which would release the NK cells from suppression by PIBF.

Preferably the vaccine comprises an adjuvant. Such an adjuvant may be but is not limited to for example Freund's mineral gels such as aluminiumhydroxide and surface active substances such as lysolecitin, pluronic polyols, polyanions, polycations (e.g. polyArg), peptides, oil emulsions, keyhole limpet hemocyanin, and dinitrophenol. Among adjuvants preferably used in humans are BCG (bacilli Calmette-Guerin) and Corynebacterium parvum. Preferably the PIBF or the derivative thereof or the fragment thereof is recombinant and a chemically synthesized molecule, respectively.

An advantageous aspect of the present invention concerns the use of a polynucleotide encoding PIBF or a derivative thereof or a fragment thereof or PIBF-antisense molecule for the preparation of an anti-tumor medicine. In the scope of the present application the term “polynucleotide encoding PIBF” or “nucleotide sequences complementary to PIBF-mRNA” relates to a sequence derived from a sequence preferably selected from the group consisting of SEQ ID No. 3, 5, 7, 9, 11, 12, 13, 16, 18, 21, 22, 24, 26, 28, 30, 33, 35, 37 or fragemnts or derivatives thereof.

Genes encoding PIBF can be turned off by transforming a cell or tissue with expression vectors which express high levels of a polynucleotide or a derivative thereof or a fragment thereof which encodes PIBF. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

Modifications of gene expression can be obtained by designing antisense molecules, DNA, RNA, or PNA, to the control regions of the gene encoding PIBF, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. The antisense molecules may also be designed to block translation of mRNA by preventing the transcript from binding to ribosomes.

The term “antisense” as used herein refers to nucleotide sequences which are complementary to a specific DNA or RNA sequence. Antisense molecules may be produced by any method including synthesis by ligating the gene(s) of interest in a reverse orientation to a viral promoter which permits the synthesis of a complementary strand. Once introduced into a cell this transcribed strand combines with natural sequences produced by the cell to form duplexes. These duplexes then block either the further transcription or translation.

In one aspect, antisense molecules to the polynucleotide encoding PIBF may be used in situations in which it would be desirable to block the transcription of the mRNA. In particular, cells may be transformed with sequences complementary to polynucleotides encoding PIBF. Thus, antisense molecules may be used to modulate PIBF activity, or to achieve regulation of gene function. Such technology is now well known in the art, and sense or antisense oligomers or larger fragments, can be designed from various locations along the coding or control regions of sequences encoding PIBF.

Expression vectors derived from retroviruses, adenovirus, herpes or vaccinia viruses, or from various bacterial plasmids may be used for delivery of nucleotide sequences to the targeted tumorous organ, tissue or cell population. Methods which are well known to the person skilled in the art can be used to construct recombinant vectors which will express antisense molecules complementary to the polynucleotides of the gene encoding PIBF.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can specifically and efficiently catalyze endonucleolytic cleavage of sequences encoding PIBF.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding PIBF. Such DNA sequences may be incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include but are not limited to the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient (allogeneic stem cell transplantation). Delivery by transfection and by liposome injections may be achieved using methods which are well known in the art.

Any of the anti-tumor medicines described above may be applied to any suitable subject including, for example, mammals such as dogs, cats, cows, horses, rabbits, monkeys, and most preferably, humans.

A further aspect of the present invention is a method for treating a patient with a tumor said method comprising administering to the patient an effective amount of an anti-PIBF antibody or a fragment thereof.

In two publications (Szekeres-Bartho et al., Am.J.Reprod.Immuno. 24, 105, 1990; Szekeres-Bartho et al., Cell.Immunol.177, 194, 1997) it was demonstrated that addition of neutralizing anti-PIBF antibody interferes with a successful pregnancy outcome in mice. Moreover, PIBF isolated from culture supernatants of progesterone treated murine lymphocytes when injected in vivo, prevented the abortive effect of anti-progesterone drugs. These data suggest that these reagents could act in a similar way in patients with cancer or autoimmune diseases.

Another aspect of the present invention concerns a method for the treatment of a tumor in a patient said method comprising administering an effective amount of PIBF or a derivative thereof or a fragment thereof.

Another preferred aspect of the present invention relates to a method for the treatment of a tumor in a patient said method comprising administering an effective amount of a polynucleotide encoding PIBF or a derivative thereof or a fragment thereof or PIBF antisense molecule.

Another aspect of the present invention refers to a pharmaceutical preparation for the treatment of a tumor in a patient said preparation comprising anti-PIBF antibody or a fragment thereof, PIBF or a derivative thereof or a fragment thereof, and polynucleotide encoded PIBF or a derivative thereof or a fragment thereof or PIBF antisense molecule, respectively. Of course, here again, the same definitions and preferred embodiments as mentioned above apply.

The pharmaceutical preparation may be administered alone or in combination with at least one other agent such as a stabilizing compound which may be administered in any sterile biocompatible pharmaceutical carrier including but not limited to saline, buffered saline, dextrose and water. The pharmaceutical preparations may be administered to a patient alone or in combination with other agents, drugs or hormones. The pharmaceutical preparations utilized for the method for the treatment of a tumor in a patient may be administered by any number of routes including but not limited to oral intravenous, intramuscular, intra-arterial, intramedullary, intrathecal, intraventricular, transdermal, subcutaneous, intraperitonial, intranasal, enteral, topical, sublingual or rectal means.

In addition to the active ingredients these pharmaceutical compositions may comprise suitable pharmaceutically acceptable carriers comprising excipients and auxiliaries which facilitate processing of the active compounds into preparations which can be used pharmaceutically. The carriers enable the pharmaceutical preparations to be formulated as tablets, pills, dragees, capsules, liquids, gels, syrups, slurries, suspensions and the like.

According to another aspect, the present invention also relates to a recombinant protein with a PIBF activity according to SEQ.ID.NO 1 and derivatives thereof. This PIBF protein according to the present invention has full PIBF activity comparable to natural PIBF (comprising the sequence according to SEQ.ID.NO 2) but does not show the exact amino acids 595 to 614 as well as amino acid No. 333 according to the natural PIBF sequence (SEQ.ID.NO 2). The protein sequence of the recombinant PIBF protein according to the present invention (SEQ.ID.NO 1) comprises 757 amino acid residues. The present invention therefore provides for novel recombinant PIBF proteins having the sequence of SEQ.ID.NO 1 or derivatives or homologs thereof. Therefore, the recombinant protein with a PIBF activity according to the present invention comprises

the amino acid sequence according to SEQ.ID.NO 1 or

an amino acid sequence with an amino acid identity of at least 98% to the sequence according to SEQ.ID.NO 1 as determined by FAST/A algorithm or

an amino acid sequence from amino acid identity of at least 95% to the sequence from amino acid residue 580 to 630 of SEQ.ID.NO 1 as determined by FAST/A algorithm and

a PIBF activity of at least 50% of the natural human PIBF molecule.

The PIBF activity may be defined and quantified as NK or-CTL inhibition. NK inhibition is considered when in the presence of PIBF the otherwise efficient effector cells (tested in the absence of PIBF) are paralized, that is either the recognition and binding (conjugation) or killing of the target cells is reduced as a function of PIBF concentration. The activity can be expressed as percentage inhibition/pg PIBF or similar substances compared to no PIBF. This similarly applies to CTL inhibitory activity (Szekeres-Bartho et al., Cell.Immunol.177 (1997), 194-199), Szekeres-Bartho et al., Am.J.Reprod.Immunol. 24, 105, (1990)). Furthermore, the PIBF activity may be defined and quantified as Th2 enhancement which is measured by quantifying Th2 (IL-3, IL-4, IL-6, IL-10) vs. Th1 (IL-12, IFN-g) lymphokines, either at the protein or at the mRNA level, and then taking the ratio of the Th2 vs. Th1 signals. An increase in the Th2 and a concomitant decrease in the Th1 cytokines indicate a Th2 enhancement. It can be expressed as an increase in the percentage of Th2 cytokine positive or a decrease in the percentage of Th1 cytokine positive peripheral blood mononuclear cells (PBMCs)/μg PIBF. It is also appropriate to measure the absolute amounts of these cytokines (according to standard methods from the literature) secreted into the culture supernatant or into body fluids as a function of PIBF concentration. The cytokine mRNAs can be measured by standard quantitative RT-PCR based assays, Szekeres-Bartho et al., AJRI 35 (1996), 348-351, Szekeres-Bartho et al., Am.J.Reprod.Immunol. 23, 26, (1990), Szekeres-Bartho et al., Am.J.Ob.Gyn. 163, 1320, (1990).

Despite the significant differences in amino acid sequence of the PIBF protein according to the present invention compared to the natural human PIBF sequence it is possible to produce a recombinant protein with a sequence as defined above (SEQ.ID.NO 1) which recombinant protein shows particularly high functional similarities to the natural protein.

According to a preferred embodiment of the present invention the recombinant protein comprises an amino acid sequence as given from amino acid residues 300 to 350 in SEQ.ID.NO 1. Amino acid No. 333 in the natural human PIBF protein (SEQ.ID.NO 2) is Cys instead of Arg in the recombinant PIBF protein according to the present invention (SEQ.ID.NO 1). Therefore, the recombinant protein according to the present invention preferably comprises an Arg as amino acid No. 333 according to SEQ.ID.NO 1 and a considerable PIBF activity (>50%). However, it may comprise on either one or both ends further amino acid residues which are identical to, homologue to or different from the amino acid residues in SEQ.ID.NO 1 as long as the recombinant protein shows a PIBF activity of at least 50% of the natural human PIBF molecule.

According to a preferred embodiment of the present invention the recombinant protein comprises an amino acid sequence as given from amino acid residues 580 to 630 in SEQ.ID.NO 1 and a considerable PIBF activity (>50%). This recombinant protein therefore comprises the sequence of the inventive PIBF between amino acid residues 580 to 630 in SEQ.ID.NO 1. It may further comprise on either one or both ends further amino acid residues which are identical to, homologue to or different from the amino acid residues in SEQ.ID.NO 1 as long as the recombinant protein shows a PIBF activity of at least 50% of a natural human PIBF molecule.

According to a further aspect of the present invention a protein with a PIBF activity comprising

the amino acid sequence according to SEQ.ID.NO 4 or

an amino acid with an amino acid identity of at least 85%, preferably at least 90%, still preferred at least 95%, most preferred 99%, to the sequence according to SEQ.ID.No 4 as determined by FAST/A algorithm

is provided. This protein has shown to be a 89-kDA protein with a PIBF activity isolated from a mouse. This amino acid sequence is particularly advantageous with respect to aspects of detecting, diagnosing and analyzing tumors, anti-tumor substances, carcinogenic substances in mice but also other laboratory animals. Furthermore, with the help of this inventive protein tests can be carried out on animals, e.g. mice, guinea-pigs, hamsters, rats, with a predisposition for a tumor. Another aspect relates to animals, in particular mice, in which this protein is inhibited or its activity blocked. This may be carried out for example by providing analogues of binding partners of this protein.

A preferred aspect of the present invention relates to a protein comprising an amino acid sequence with an identity of at least 85%, preferably at least 90%, still preferred at least 95% as determined by FAST/A algorithm to a sequence selected from the group consisting of SEQ.ID.NOs 6, 8, 10, 14, 15, 17, 19, 20, 23, 25, 27, 29, 31, 32, 34 and 36 said protein being an alternatively processed PIBF protein. It has been shown that alternatively processed mRNA molecules are present in different tissues and therefore express also alternatively processed proteins. Surprisingly, these alternatively processed proteins are present in tumor tissues at another concentration compared to the healthy tissues. This can be advantageously used for detecting and analyzing tumors in a sample whereby particularly SEQ.ID. NOs 6 and 8 are preferred since these are two smaller PIBF forms found in human primary tumors, SEQ.ID.NO 6 in gastric adenocarcinoma and SEQ.ID.NO 8 in endometrial adenocarcinoma. Normal tissue counterparts from the same patients did not express detectable levels of these PIBF-mRNA splice variants. It has, however, also been shown that exons 17 and 18 are included in almost all forms identified. Therefore, peptides comprising these exons are particularly advantageous. The term “alternatively spliced PIBF proteins” refers to proteins which are derived from proteins with PIBF activity.

According to a further aspect the present invention provides a nucleic acid molecule encoding the above described recombinant protein with a PIBF activity according to the present invention. Of course, it is further possible for the nucleic acid molecule to comprise an additional sequence encoding at least one second protein other than the PIBF protein thereby providing a nucleic acid sequence encoding a fusion protein comprising at least in one part a peptide with PIBF activity.

With respect to the mouse nucleic acid molecule, e.g. SEQ ID No 5 or a fragment thereof, this is preferably used to produce knock out mice, e.g. mice, in which the expression of the PIBF gene or a fragment thereof is blocked or inhibited. This will for example be carried out by providing an antisense mouse PIBF nucleic acid molecule or fragment thereof which strategy is described above. Another aspect of the present invention therefore relates to knock out mice which show an inhibited or reduced expression of active PIBF protein.

Preferably, a nucleic acid molecule encoding an alternatively processed PIBF protein are provided comprising a nucleic acid sequence with an identity of at least 80%, preferably at least 90%, still preferred at least 95% to a sequence elected from the group comprising SEQ.ID. NOs 7, 9, 11, 12, 13, 16, 18, 21, 22, 24, 26, 28, 30, 33, 35 and 37 or

a sequence which hybridizes under stringent conditions to one of the above sequences or

a sequence which is degenerated due to the genetic code of one of the above sequences.

These are nucleic acid sequences which correspond to alternatively spliced mRNA molecules found in various tissues whereby particularly SEQ.ID. NO 7 and SEQ.ID.NO 9 relate to alternatively spliced mRNA molecules which were found only in tumor tissues, however normal tissues did not comprise these mRNA sequences. Therefore, particularly these are advantageous when detecting or analyzing tumors as well as healthy cells and tissues.

According to a further aspect the present invention relates to a nucleic acid vector comprising an inventive nucleic acid sequence.

When the above mentioned vector according to the present invention is introduced into a suitable host mRNA is produced which provides an RNA strand for the translation of a recombinant protein with PIBF activity according to the present invention or an inventive protein.

The regulatory element can be any suitable element known by the skilled person in the art in particular a specific promotor which is chosen in accordance with the specific host into which the vector is to be introduced in order to achieve a maximum production of recombinant protein. The regulatory element can further comprise enhancers which enhance the transcription.

Preferably the nucleic acid vector further comprises a selection marker. The selection marker can be any suitable marker which is well-known by the skilled person in the art in order to select cells or host organisms into which the vector has been introduced. Such selection markers may be for example, any gene encoding an antibiotic resistance conferring protein, or a gene encoding a protein necessary for the cell metabolism, whereby the cells or host organisms in which the above mentioned vector is to be introduced show a deficiency for this protein. The selection marker may further be any gene which will change the phenotype of the cell or host organism which has taken up the above mentioned vector, e.g. the colour.

According to a further aspect the present invention relates to a cell comprising the above mentioned vector according to the present application. The vector may be integrated into the genome of the cell or also present as exogenous DNA in the cytoplasma as long as transcription of the complementary nucleic acid molecule is provided. In the scope of the present invention the term “cell” comprises any procaryotic or eucaryotic cell. These cells will be preferably used to produce recombinant proteins with PIBF activity according to the present invention. These produced recombinant proteins can be isolated and purified according to methods well known in the art and used further, e.g. for the production of pharmaceutical preparations comprising recombinant proteins with PIBF activity according to the present invention.

The invention will be described in more detail by the following examples and figures but the invention is of course not limited thereto.

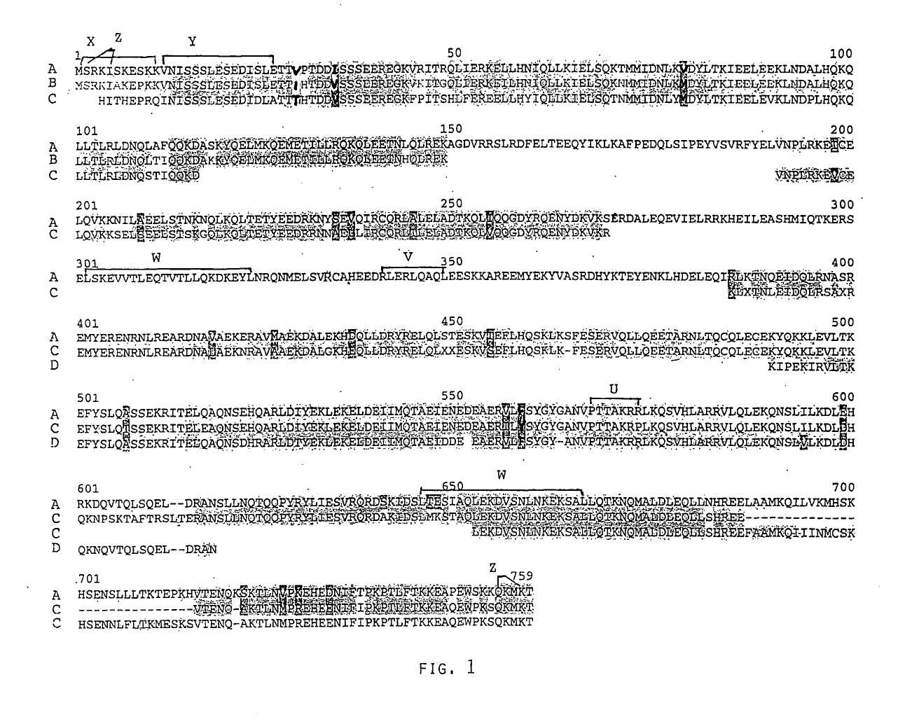

FIG. 1 shows an alignment of recombinant and (natural) mouse PIBF amino acid sequences,

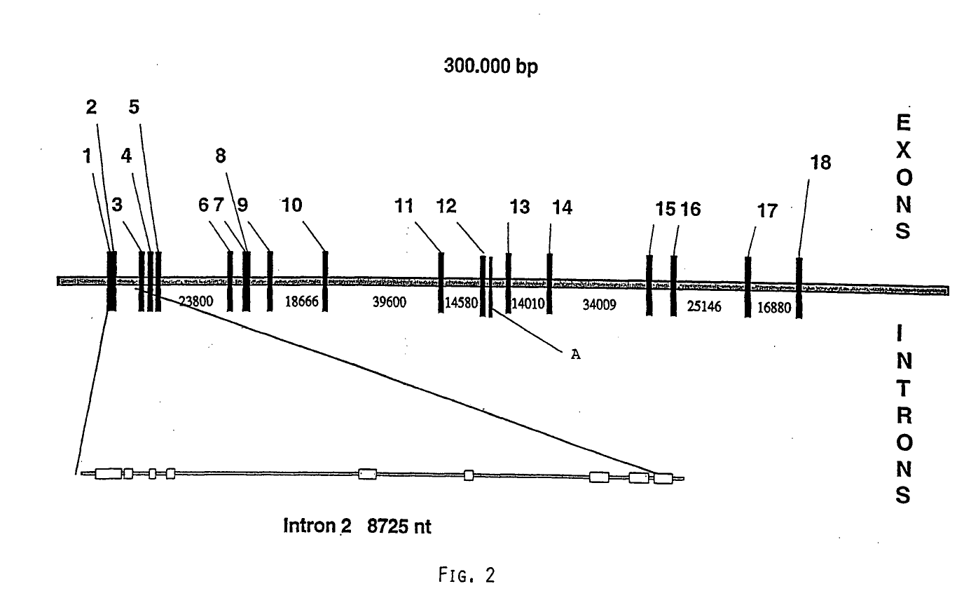

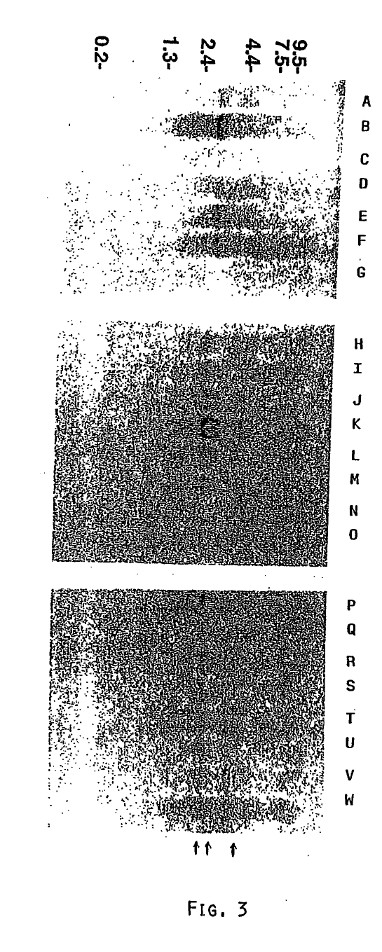

FIG. 2 shows a schematic representation of the exons and introns in the PIBF gene region on chromosome 13,

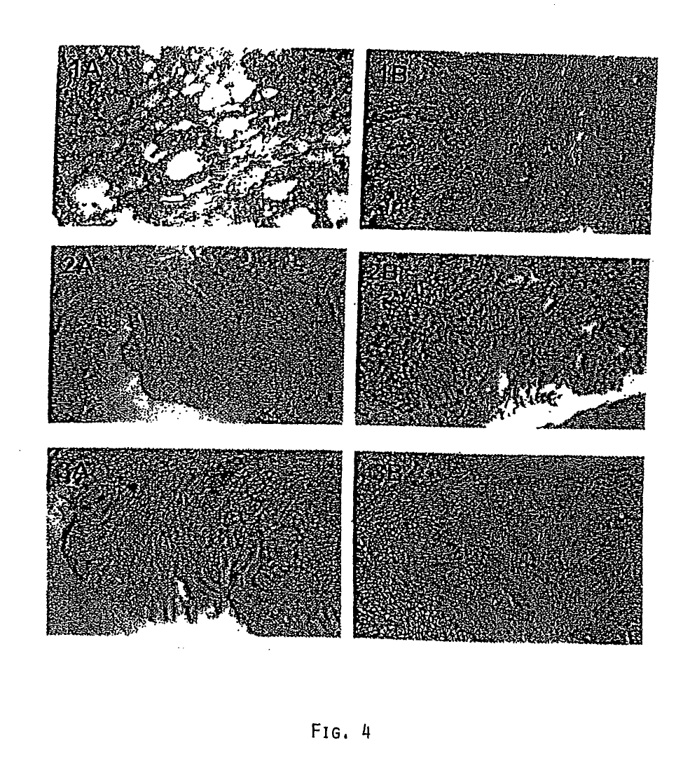

FIG. 3 shows a northern blot for the detection of PIBF-mRNA in different tissues.

FIG. 4 shows the immunohistochemical analysis of human primary tumors.

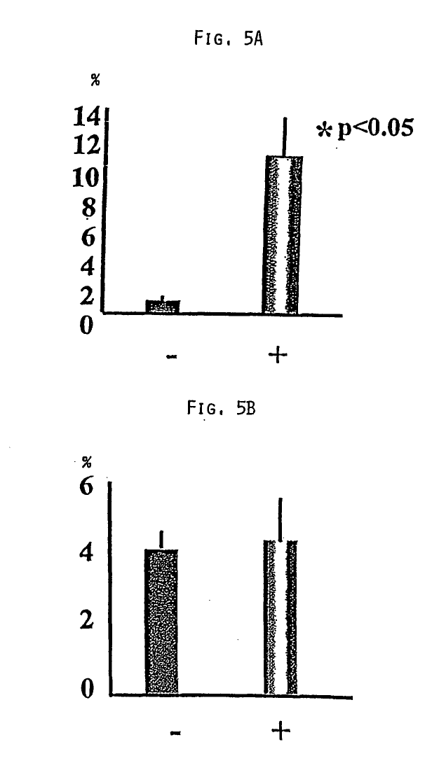

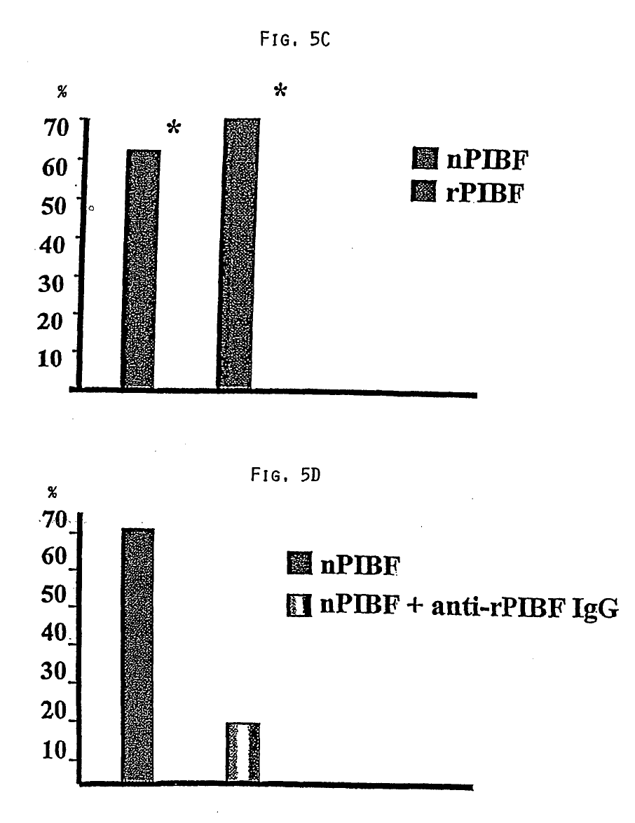

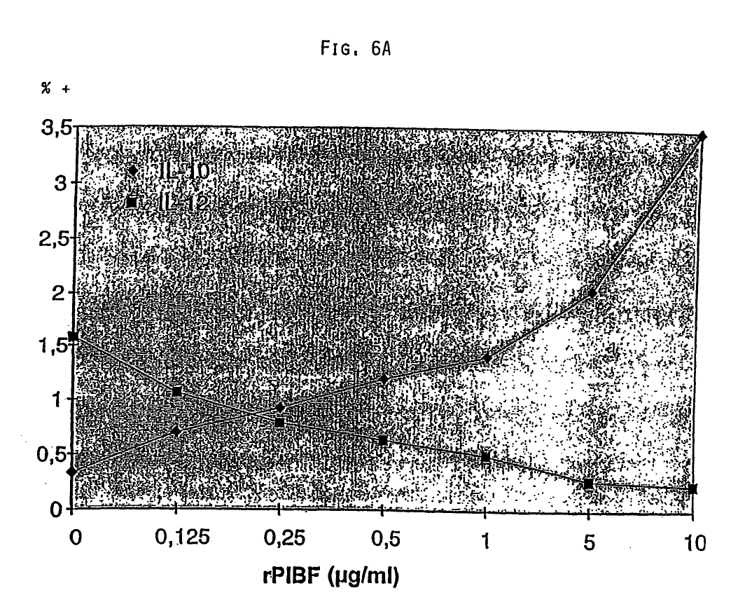

FIGS. 5A-5D show the influence of anti-PIBF treatment on NK cell target killing of tumor cells.

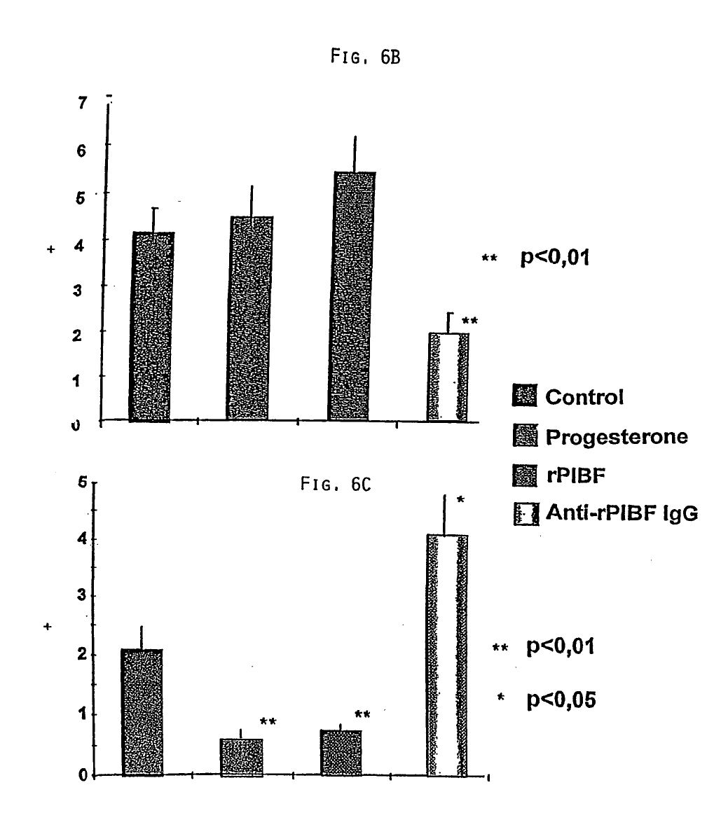

FIGS. 6A-6C show the effect of the recombinant PIBF on IL-10 and IL-12 expression of non-pregnancy lymphocytes.

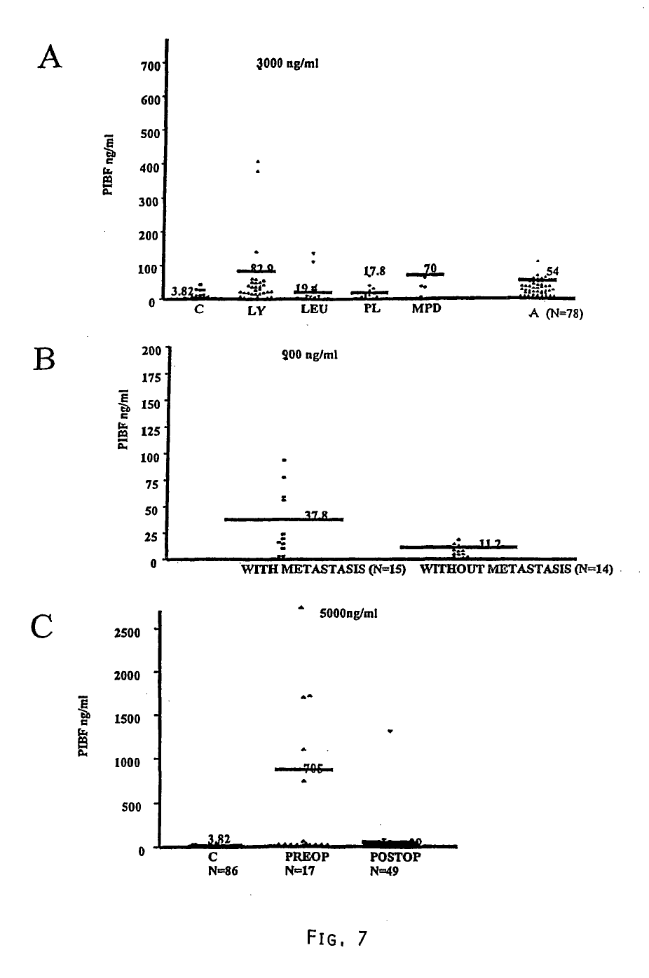

FIG. 7 shows PIBF levels in urine samples of patients with non-adenocarcinoma tumors and non-solid tumors.

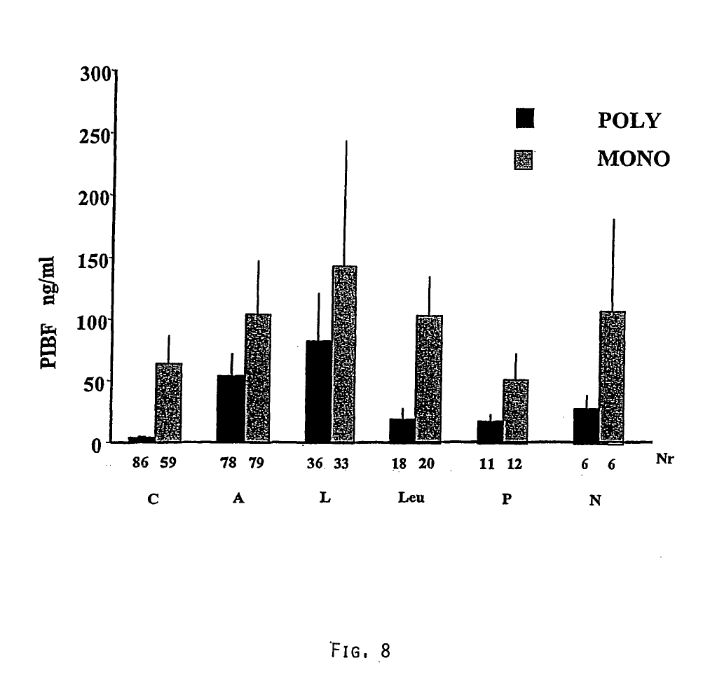

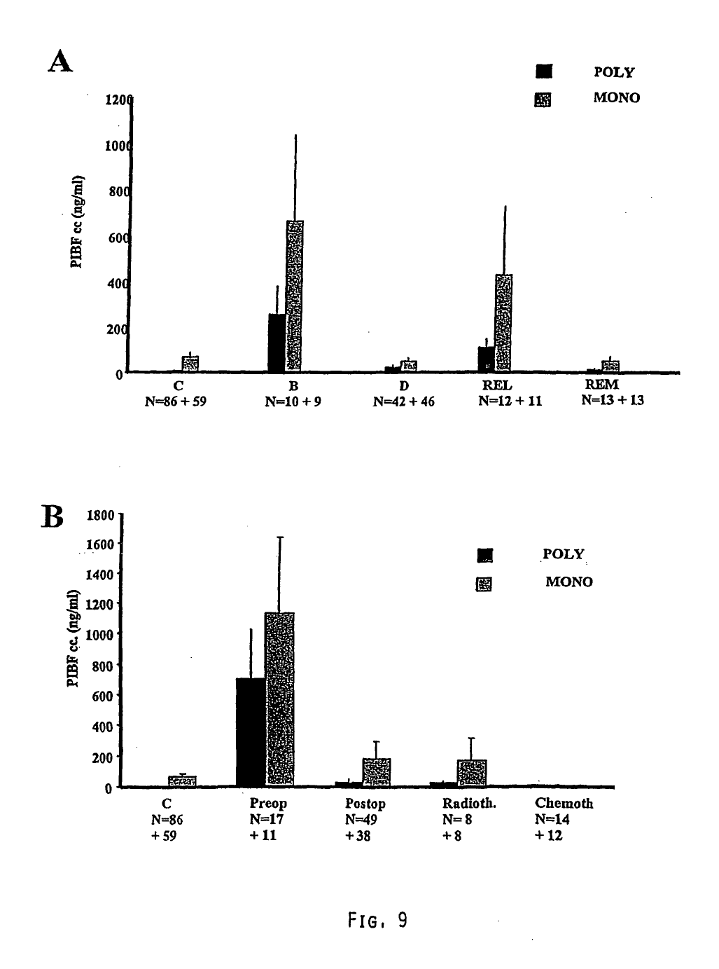

FIG. 8 shows the detection of elevated PIBF levels with the help of monoclonal and polyclonal antibodies.

FIG. 9 shows the normalization of PIBF levels after surgery or chemotherapy.

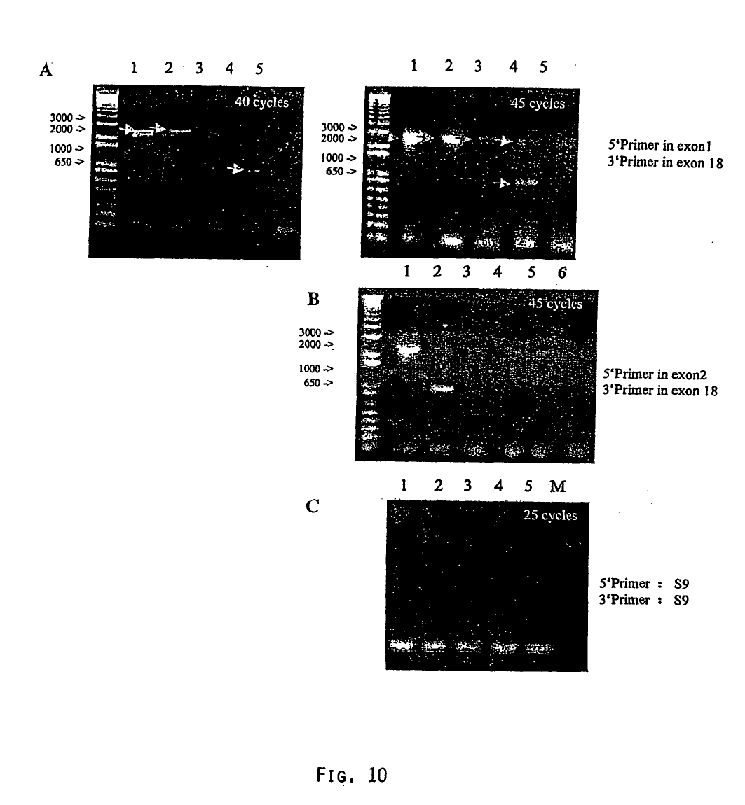

FIG. 10 shows the different PIBF-mRNAs overexpressed in human primary tumors.

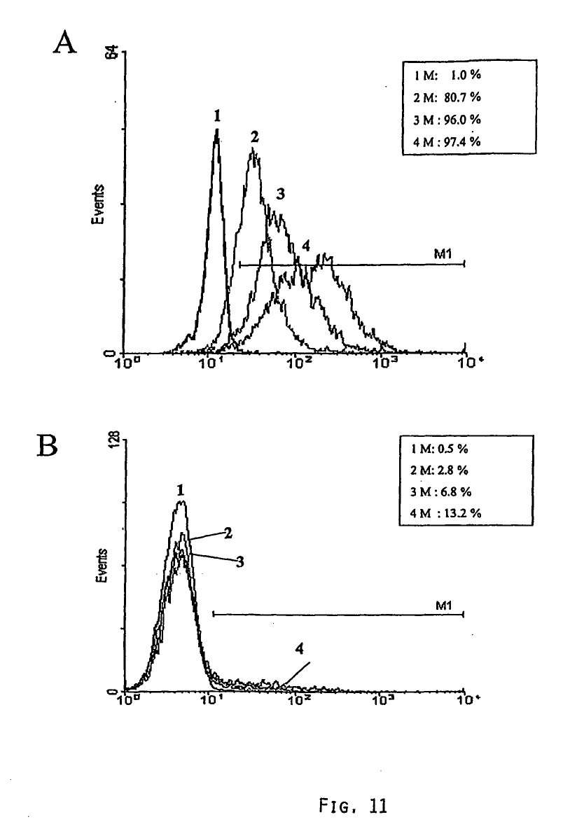

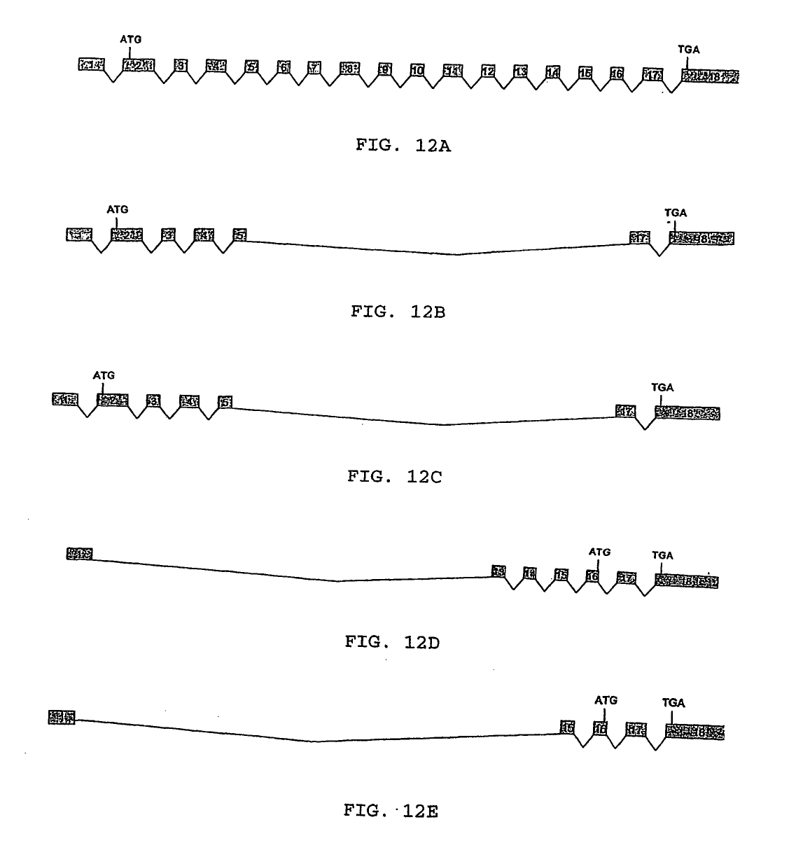

FIG. 11 shows the PIBF binding to human PBMC.

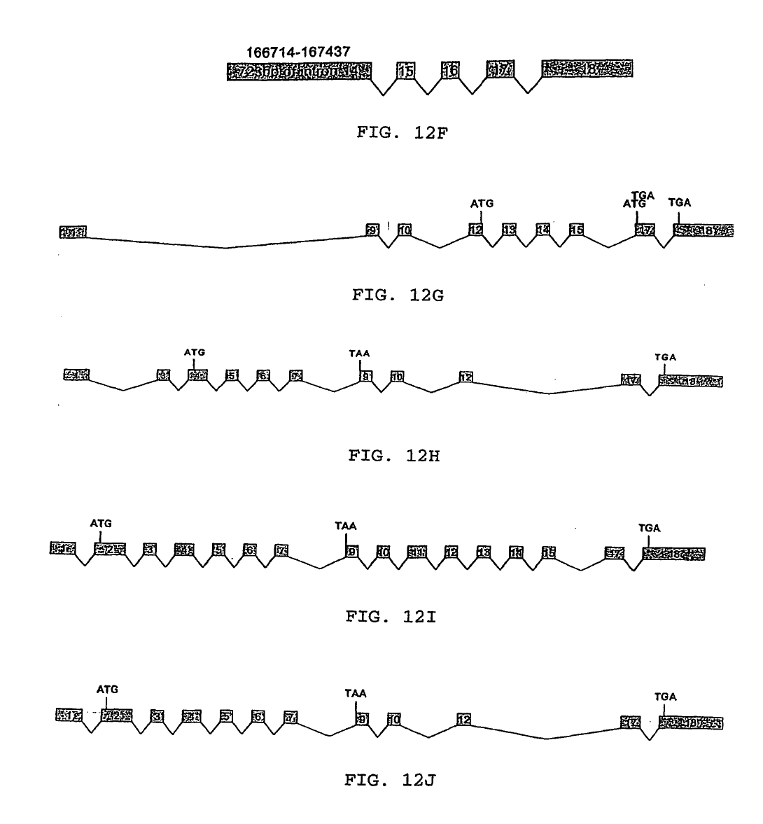

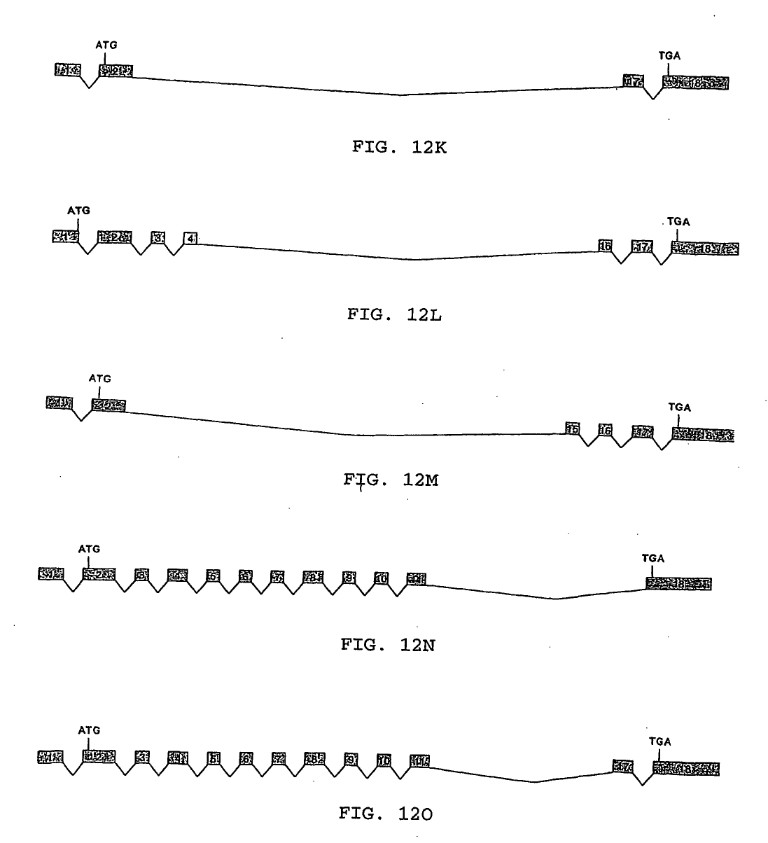

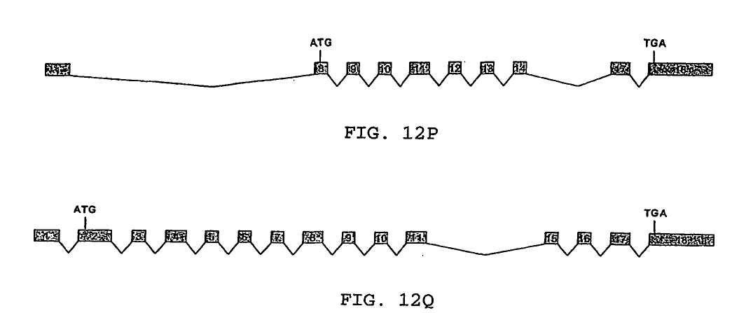

FIG. 12 shows alternatively spliced PIBF-mRNA.

FIG. 1 shows the alignment of recombinant (human) and mouse (natural) PIBF, A being the recombinant sequence, B the IC mouse sequence (cloned from mouse testis library), C being the EST mouse, pieced together from dEST libraries based on the human sequences, and D being the bovine sequence. X represents the signal sequence according to the PSG prediction method, y the signal sequence according to the GvH prediction, z the ER membrane retention signal, w the leucine zipper pattern—DNA binding motif, v the peroxisomal targeting signal, and u the nuclear localisation signal.

The PIBF gene is located to chromosome 13. A number of introns are present in the PIBF gene (s. FIG. 2), whereby in intron 2 there are multiple copies of the Alu repeat element which serves as a site for alternative splicing. A shows a gap between genomic contigs.

FIG. 3 shows a nothern blot for the detection of PIBF-mRNA in various normal tissues: stomach (A), thyroid gland (B), spinal cord (C), lymph node (D), trachea (E), adrenal gland (F), bone marrow (G), spleen (H), thymus (I), prostate (J), testis (K), uterus (L), small intestine (M), colon (N), PBL (O), heart (P), brain (Q), placenta (R), lung (S), liver (T), skeletal muscle (U), kidney (V), pancreas (W). The arrows in FIG. 4 indicate 3 different mRNA forms.

### EXAMPLE 1

### ESTs Entries Matching the Human PIBF Sequence

ESTs entries in human cDNA libraries were searched which match the human PIBF sequence. 43 entries having PIBF sequences were found out of 2.2 million dESTs deposited in 3776 human cDNA libraries. These 43 entries belong to 27 different libraries. 7 of the 27 (25%) libraries originate for normal (non-pregnant, non-tumors adult) tissues. Importantly, testis, which is a immunoprivileged tissue, frequently indicated the presence of PIBF mRNA. 13 of the 27 libraries contain mRNAs expressed in tumorous tissues (˜50%). The rest is from foetal or pregnant tissues. This shows that PIBF is preferentially expressed during development, pregnancy and malignancy. However, the number of matching ESTs can correlate with the mRNA abundance but heavily depend on the quality of the library. For that reason one can not take it as a measure of expression level directly (see table I).

TABLE I

| | | Origin of cDNA library | | |
|---|---|---|---|---|
| | ACC # | Organ | Embyonic, normal, tumor | Maching parts (nt) |
| 1 | AA099685 | Uterus | Normal, pregnant | 860-1321 |
| 2 | AI188926 | Placenta, pooled (2) | Normal, 8-9 weeks pregn. | 2409-2763(87) |
| 3 | AI200713 | Placenta, pooled (2) | Normal, 8-9 weeks pregn. | 2418-2763(86) |
| 4 | N27300 | Placenta, pooled (2) | Normal, 8-9 weeks pregn. | 2367-2763(84) |
| 5 | N40036 | Placenta, pooled (2) | Normal, 8-9 weeks pregn. | 1657-2089(20) |
| 6 | AA251149 | Tonsilla, germ. center | Normal - enriched B cells | 2440-2763(84) |
| 7 | AA251594 | Tonsilla, germ. center | Normal - enriched B cells | 307-633 |
| 8 | AA806027 | Tonsilla, germ. center | Normal - enriched B cells | 1644-2025 |
| 9 | AA610068 | testis | normal | 2455-2763(86) |
| 10 | AI126269 | testis | normal | 2385-2763(91) |
| 11 | AI758409 | Kidney, bulk tissue Kid11 | Normal | 2491-2763(83) |
| 12 | H64996 | Nose (Olfac epithel) | Normal (female) | (75)1669-1837(98) |
| 13 | AW793587 | Uterus (exp ORFs) L | adult | (224)1989-2346 |
| 14 | AW818553 | Stomach ORF | adult | 2544-2667(8) |
| 15 | BE165549 | Head-neck | adult | 1881-2264 |
| 16 | AA913693 | Lung-testis-B cell | normal + fetal (lung) | 2501-2763(8) |
| 17 | AA971010 | Lung-testis-B cell | normal + fetal (lung) | 2531-2763(102) |
| 18 | AI014561 | Lung-testis-B cell | normal + fetal (lung) | (41)1616-2116 |
| 19 | AI222385 | Lung-testis-B cell | normal + fetal (lung) | 2361-2763(8) |
| 20 | AI809069 | Lung-testis-B cell | normal + fetal (lung) | 1644-2179 |
| 21 | AW085186 | Lung-testis-B cell | normal + fetal (lung) | 2328-2763 (86) |
| 22 | AW269537 | Lung-testis-B cell | normal + fetal (lung) | 2376-2763(83) |
| 23 | AW572968 | Lung-testis-B cell | normal + fetal (lung) | 2515-2616 |
| 24 | AI350620 | whole body | Fetus (8-9 weeks) | 2565-2763(87) |
| 25 | D31319 | Lung, bulk tissue | fetal | 1394-1765 |
| 26 | AA004593 | Liver + spleen | Fetal (20 weeks) | (175)900-1019(54) |
| 27 | AI741044 | 5 pooled libaries* | Fetal, placenta, tumor | 2349-2763(95) |
| 28 | AI808795 | 5 pooled libaries* | Fetal, placenta, tumor | 2406-2725(146) |
| 29 | AW978222 | Colon | Tumor, metastasis | 1656-2135 |
| 30 | AI254231 | Colon | adenocarcinoma | (140)2482-2763(101) |
| 31 | AA307364 | Colon, cell line | Carcinoma (HCC) | 2017-2391(55) |
| 32 | AA603710 | Germ cell | Mixed tumors | 2404-2763(84) |
| 33 | AI350870 | Germ cell | Mixed type tumors (3) | 2511-2763(84) |
| 34 | AI990811 | Germ cell (GC_6) | Pooled tumors | 2283-2763(96) |
| 35 | AI278790 | Lung, neuroendocrin | Carcinoid | 2514-2763(92) |
| 36 | AI554801 | Uterus, pooled (2) Ut3 | Endometrial carcinoma | 2407-2763(88) |
| 37 | AI915158 | Uterus, pooled (3) (ut2) | Serous papill cc. high gr | 1714-1837(109) |
| 38 | AW273347 | Uterus (pooled (2) | Serous papill cc. high gr | 1785-2311(50) |
| 39 | AW169084 | Uterus, pooled (3) (ut2) | Endom carcinoma | 2200-2763(84) |
| 40 | AI769755 | Kidney pool of 2 Kid12 | Tumor, clearcell | 2671-2763(403) |
| 41 | AW769371 | Kidney (pool of 2) Kid13 | Wilms tumors (pr, + meta) | 2310-2763(102) |
| 42 | N59340 | Brain (male) | Mult. Scler. lesion | (102)2506-2763(83) |
| 43 | N77149 | Brain (male) | Mult. Scler. lesion | 1858-2175(12) |

### EXAMPLE 2

### Determination of PIBF Concentration in Urine Samples from Cancer Patients

A competitive ELISA-based assay to measure PIBF in the urine of cancer patients was established. ELISA plates were coated with recombinant human PIBF at a concentration of 2 μg/ml. Biotin labelled polyclonal anti-PIBF IgG was added together with the samples to be determined for PIBF content. The higher is the PIBF concentration in the sample, the lower is the corresponding ELISA value. Based on these ELISA readings the absolute concentration of the PIBF can be determined.

Urine samples were collected from cancer patients, and used fresh or frozen shortly after collection and stored at −20° C. until analysis. It was determined previously that PIBF levels in the serum are significantly higher in healthy pregnant women relative to non-pregnant or pathologic pregnancy levels. To validate the assay on urinary PIBF from cancer patients, urine samples from healthy pregnant women and normal healthy non-pregnant individuals served as positive and negative controls, respectively. The results are summarized in Table II. Normal (healthy, non-pregnant) individuals have low urinary PIBF concentrations (5 ng/ml). Urine from pregnant women was characterized by an average of 110 ng/ml PIBF concentration. Importantly, the high PIBF levels. quickly returned to normal following abortion or labor. Analysis of urine samples from 65 tumor patients clearly showed that tumor bearing patients had significantly higher amount of PIBF in their urine, than healthy non-pregnant individuals, ranging from 5 to 180 ng/ml. Patients with advanced cancer (big size primary tumor and/or metastasis) seemed to have higher values, examplified by the data on urine samples from lung tumor patients having an average of 28 vs. 43 ng/ml concentration with or without metastasis, respectively.

This data indicates that PIBF concentration is related to tumor mass, and detection of PIBF in the urine can be used for monitoring of disease progression and relapses. The rise in urine PIBF concentration as a consequence of the presence of PIBF producing tumor is even more pronounced, since not all tumor types are PIBF positive (˜70-80% of tumors tested so far).

The most prevalent tumor type among the patients was lung cancer with 23 cases. Most of the lung cancer patients had high PIBF concentration. The lack of high PIBF in the urine could be correlated with clinical disease status, namely after the removal of the primary tumor and in remission PIBF concentrations were significantly lower or even normal.

TABLE II

| | | Threatened | Tumor | |
|---|---|---|---|---|
| Control | Pregnant | preterm | patients | |
| (a) | (b) | labour (c) | (e) | |
| n = 48 | n = 23 | n = 19 | n = 65 | |
| x = 5.5 +/− 1.8 | x = 110 +/− 36 | x = 6 +/− | x = 27.7 +/− 5.3 | |
| | | 4.7 | Lung (n = 23) | |
| a − b p < 0.001 | b − c < 0.02 | | Without | With |
| | | | metastasis | metastasis |
| | | | (n = 12) | (n = 11) |
| | | | 29.4 | 43.1 |
| a − c NS | | | | |
| a − d p < 0.001 | | | | |
| a − e p < 0.001 | | | | |

### EXAMPLE 3

### Detection of PIBF in Tumor Tissues

Following the demonstration of PIBF expression by MCF-7 (human mammary epithelial carcinoma) cell line, a series of human primary tumors were investigated for the expression of PIBF. It became obvious that PIBF appears in the culture supernatant of MCF-7 cells, suggesting that this protein is expressed and secreted similarly what was found with pregnant or activated lymphocytes in culture. According to proteomic analysis, anti-PIBF antibodies recognize proteins with two different sizes in the cellular lysate by 2D Western analysis. A 34-kDa spot is likely to correspond to the secreted form. Another major, 60-62 kDa doublet is detected, which might be the major cell associated PIBF form.

A variety of formalin-fixed human primary tumors were analyzed ex vivo by immunohistology using polyclonal rabbit anti-serum generated by immunization with human natural PIBF (34-kDa) or recombinant PIBF (89-kDa). The results show that a lot of tumor types tested express PIBF or PIBF related substances, e.g. PIBF molecules of different lenghts, PIBF molecules from differently spliced mRNA, truncated molecules, fusion proteins, etc. (Table III). 15 out of the 27 tumors (55%) showed strong positive staining. The lack of specific immunostaining of the normal tissue counterparts proves that transformed tumor cells differentially express PIBF (see FIG. 4). In FIG. 4 left column, designated “A”, the normal tissue is shown, in the right column, designated “B”, tumor tissues are shown. In the first row (designated “1”) lung cancer (small cell), in the second row (designated “2”) urinary bladder carcinoma (transitorial cell), and in row “3” stomach cancer (adenocarcinoma) are shown. These data also substantiate that PIBF positivity is a result of expression by the tumor cells themselves, and not the binding of PIBF from the extracellular fluid (secreted by infiltrating lymphocytes).

TABLE III

| | | No of PIBF |
|---|---|---|
| | Tissue and Tumor | positive |
| Organ | type | (No of tested) |
| Stomach | Adenocarcinoma | 1 (2) |
| Gall bladder | Adenocarcinoma | 0 (1) |
| Prostate | Adenocarcinoma | 0 (1) |
| Colon | Adenocarcinoma | 1 (1) |
| | Primaer tu. | |
| Ovarium | Cystadenocarcinoma | 2 (3) |
| Thyroid gland | Carcinoma papillare | 2 (2) |
| Breast | Invasive ductal cc | 1 (1) |
| Breast | Invasive lobular cc | 1 (1) |
| Uterus | Cc. endometrioides | 0 (1) |
| Uterus | stromal carcinoma | 0 (1) |
| Uterus | Leiomyosarcoma | 0 (1) |
| Septum nasi | Leiomyosarcoma | 0 (1) |
| Skin | Melanoma | 0 (2) |
| Skin | Epithelial cc. | 1 (1) |
| Lung | Epithelial cc. | 2 (2) |
| Oesophagus | Adenocarcinoma | 1 (1) |
| Urinary bladder | Transitorial cell cc. | 1 (1) |
| Metastasis (skin) | Kidney clear cell cc. | 0 (1) |
| Metastasis (lymph node) | Squamous epithelium | 1 (1) |
| Metastasis (lymph node) | Adenocc. coli | 1 (2) |

Based on the above mentioned results PIBF production is a quite general phenomenon of the malignant or undifferentiated state, and as a consequence, PIBF can serve as a tumor marker.

### EXAMPLE 4

### Modulating NK Activity by the Presence of PIBF

Taken all the data together on the potential importance of PIBF in the suppression of anti-tumor responses, it is plausible that PIBF produced—secreted or cell surface expressed—by tumor cells will inhibit killer cell activity systemically or locally. It has been long known that there are cell lines which are good targets in NK assays, others are not. The human tumor cell line, MCF-7 belongs to the poor target category. It is certainly a possibility that the low killing activity against these cells is the result of PIBF production, which inhibits NK activity, since the MCF-7 cell line is shown to produce PIBF. To test this possibility, PIBF-expressing MCF-7 cells were used as targets in a 4 hours single cell cytotoxicity assay, according to Grimm and Bonavida “Frequency determination of killer cells by a single-cell cytotoxic assay”; Methods Enzymol 93, 270 (1983). In FIG. 5 it is shown that anti-PIBF treatment enhances NK cell target killing of tumor cells: The minus and the plus indicate the treatment with or without anti-PIBF IgG, the numbers are the percentage of NK activity for FIGS. 5A and 5B and the percentage of inhibition of NK activity for FIGS. 5C and 5D. The source of NK cells were freshly isolated PBMCs from healthy individuals. In fact, treatment of these tumor cells with anti-PIBF IgGs increased their sensitivity to NK-mediated lysis dramatically, approximately 8-10 fold (FIG. 5A). The basic killing activity against MCF-7 cells is very low (1-2%), whereas high values (50-80%) can be measured when K562 cells are used as target cells in parallel assays. The same anti-PIBF treatment, which seems to be effective in increasing the target activity of tumor cells, however, had no effect on a non-tumor cell line (McCoy, human embryonic fibroblast) (FIG. 5B). By characterizing representative members (good vs. poor target) from both groups, one can make a correlation between expression of PIBF and NK cell activity. Moreover, this assay allows to test the effectiveness of exogenous PIBF to reduce PIBF-target cell killing, and more importantly to evaluate the power of neutralizing anti-PIBF antibodies in stimulating the lysis of PIBF+ tumor cells. Neutralizing rabbit anti-human and also anti-mouse antisera are generated whereby these polyclonal antibodies are able to inactivate natural PIBF in vitro (human lymphocyte cultures in NK assay) or in vivo (pregnant mice, as animal model). By addition of recombinant PIBF to K562 cells (as targets) and PBMCs (as NK cell source), it was possible to reduce the basal killing activity by 60-70% (FIG. 5C). Antibodies generated against the recombinant PIBF removed that inhibition on killing activity almost completely (FIG. 5D).

### EXAMPLE 5

### Modulation of Cytokine Balance

One of the main mechanism of the pregnancy promoting action of PIBF is the induction of the T_{H2} cytokines. There is evidence now that the recombinant form of PIBF is also active to modulate cytokine expression by peripheral blood lymphocytes in vitro. To test the functionality of the recombinant human PIBF which was expressed in E. coli and purified by the GST-tag, rPIBF was added to non-pregnancy peripheral lymphocytes isolated by Ficoll-Paque gradient and cultured at 10⁶/ml cell density. The production of the prototype T_{H2} lymphokine, IL-10, was measured by detecting and counting the number of IL-10 positive lymphocytes (by immunohistochemistry on cytospins) after 24 h treatment. The percent of IL-10 positive lymphocytes increased as the function of rPIBF concentration from 0.35+/−0.15 to 3.5+/−1.5%. At the highest rPIBF concentration (10 μg/ml) 10× more IL-10 positive lymphocytes were present than in control cultures (FIG. 6A). The opposite effect was seen on IL-12 (T_{H1} lymphokine)-producing lymphocytes by the same treatment. The number of IL-12 positive lymphocytes decreased as the function of PIBF concentration resulting in an approximately 8 fold reduction at the highest amount of PIBF. Neutralization of the effect of natural PIBF on cytokine production was also successful. Treatment of pregnancy lymphocytes (producing PIBF) with anti-PIBF IgGs for 3 h resulted in a significant decrease in the number of IL-10 positive and a significant increase in the number of IL-12 positive cells (FIGS. 6B and C). These results prove that the recombinant form of PIBF is active in inducing T_{H2} cytokine expression. More importantly, neutralizing antibodies can remove active PIBF produced by cells in vivo and consequently enhance TH₁ cytokines.

### EXAMPLE 6

### Diagnostic Assay

The diagnostic value of urinary PIBF levels in malignancies is further exemplified by using urine samples of patients with non-adenocarcinoma tumors and nonsolid tumors (FIG. 7); dots represent results with individual sera. Average numbers are shown. N means number of patients.

### Examples

Different hematologic malignancies, especially lymphomas (LY, N:36), and also leukaemias (Leu, N=18), plasmocytomas (PL, N=11) and myeloproliferative diseases (MOP, N=7), are characterized by a higher than normal concentration (control C, N=86) of urinary PIBF (FIG. 7A); A=all tumors). Other examples are head and neck tumors (FIG. 7C) and the malignancies of the urinary tract (FIG. 7B) +=with metastasis, N=15; −= without metastasis, N=14. It is also obvious that mass of the tumor tissue—that is disease with or without metastasis, removal of tumor—greatly effects the PIBF concentration (FIGS. 7B, C).

The polyclonal antibodies are replaced in a similar antigen capture sandwich assay with a pair of monoclonal antibodies (ab) produced in mice using the N-terminal 48-kDa rPIBF as antigen. Approximately twenty different hybridoma clones were tested, then four stable and well producing clones selected for ELISA assay, and also for other diagnostic methods (immunohistochemistry). These hybridoma clones are deposited at the Hybridoma Cell Bank at the University Medical School of Pécs. (Deposition numbers are 11-14/2001, cell line codes: HYB255-258). The elevated PIBF levels in urine samples of tumor patients are detected with the monoclonal antibody pairs similarly to the polyclonal antibodies (FIG. 8), whereby C=control, A=all haematological tumors, L=lymphoma, Leu=leukemia, P=plasmacytoma, N=non defined; No.=number of patients; POLY=polyclonal ab, MONO=monoclonal ab.

The more excessive data also strengthen not only the diagnostic, but especially the tumor monitoring potential of urinary and serum PIBF ELISA. The test is able to detect and predict therapeutic success and failure, as it is shown by normalization of PIBF levels after surgery or chemotherapy, and a significant rise in relapsing patients (FIG. 9A:haematological, B:urinary track tumors) both with the polyclonal and the monoclonal anti-PIBF antibodies, whereby C=control, B=before treatment, REL=relapse, REM=remission.

### EXAMPLE 7

### Different Forms of PIBF

In addtion to the full length human PIBF, the mouse full length PIBF mRNA and protein sequence was identified (SEQ.ID.No 4,5). The mouse PIBF is also organized into 18 exons, and shows an amino acid homology of 89%.

During the attempt to compare PIBF mRNA levels in normal and tumor tissues with RT-PCR, a number of alternatively spliced PIBF mRNA forms were discovered. The structure of the alternatively spliced mRNAs and the corresponding protein products are summerized in Table IV. All the forms identified in one species may occur and can have similar functions in other species. It is examplified by the homologues 35-kDa PIBF protein forms. With the exception of one form of mRNA containing an alternative exon 14′ DNA which is intronic sequence in the predominant pre-mRNA, all of them were generated by perfect exon skipping missing several exons relative to the full length. The exons 17 and 18 sequences are included in almost all forms identified. It suggests that the amino acid sequences encoded by these exons are essential for PIBF function. Moreover, several mRNA forms with different sequences result in this same C-terminal PIBF polypeptide with predicted 10-kDa molecular weight. The truncated forms of PIBF identified by RT-PCR analyses of RNA samples were isolated from different human (SEQ.ID.No 6, No 10-20) and mouse tissues and cell lines (SEQ.ID.No8,9,23-37).

The different PIBF forms are differentially expressed and have different functional attributes.

### Examples

The full length mRNA (exons 1-18) encodes for a nuclear 89-kDa protein. Bioinformatics predicts a nuclear compartmentalization based on the two nuclear localization signals in exons 7 and 13, and a nucleic acid binding domain in exons 14-16. According to cell fractionation and Western blotting with monoclonal anti-PIBF antibodies, the 89-kDa protein indeed localizes exclusively to the nuclear fractions. Smaller molecular weight forms are present in the cytosolic and secreted fractions of different human and mouse primary tumors, embryo and cell lines.

The full length mRNA can be found in almost all tissues based on Northern (FIG. 3) and RT-PCR analysis (FIG. 10), however, in different amounts. Semiquantitative RT-PCR was performed on matched tumor/normal tissue pairs. The same amount of RNA was amplified with either PIBF specific exon 1/exon 18 (FIG. 10A) and exon 2/exon 18 primer pairs (FIG. 10B) or with ribosomal protein S9 specific primers (for loading control) of the same samples. The samples are: 1=placenta cDNA, 2=stomach tumor, 3=stomach normal, 4=uterus tumor, 5=uterus normal, 6=Neg. Ctrl: w/o template. Fast growing cell, e.g. in tumors and embryo, cell of immune priviledged tissues (testis, placenta) contain more full length PIBF mRNA. RT-PCR analysis of human primary tumors and subsequent cloning and DNA sequencing of PIBF cDNA reveal that the alternatively processed PIBF mRNA forms are expressed differently. It is exemplified by two smaller PIBF forms found in human primary tumors. Expression of the exons (1-5)−(17-18) form in gastric adenocarcinoma and that of the exons 1−(13-18) form in endometrial adenocarcinoma is restricted to the tumor tissues, since the normal tissue counterparts from the same patients do not express detectable levels of these PIBF mRNA splice variants. Importantly, both of these and other alternatively processed PIBF mRNAs are also found in tissues of immune priviledge (placenta, embryo, testis) and in immune cells.

The function of PIBF also depends on the structure of the mature protein from. One of the most interesting forms is encoded by exons 2-3-4-5-17-18 mRNA frequently found in human and mouse tissues, such as human and mouse placenta, human lymphocytes, mouse embryo and human gastric tumor. It encodes for a 298 and 297 amino acid long polypeptide with predicted 35-kDa molecular weight. (SEQ.ID.No 6,7 for human, SEQ.ID.No 8,9 for mouse). The two proteins are 86% homologous. FACS analysis reveals that the human 35-kDa form binds specifically to human immune cells (FIG. 11): FACS staining of human PBMCs with 35-kDa PIBF and α-PibF antibodies (anti-rabbit-FITC). Cells are shown in the monocyte cell region (FIG. 11A) and in the lymphocyte gate (FIG. 11B). M is the number of cells in the increased fluorescence gate expressed in percentage (%), the numbers refer to: 1=PBMC+anti-exon 17 PIBF, 2=PMBC+PIBF-35 kDa (1 μg)+anti-exon 17, 3=PBMC+PIBF-35 kDa (5 μg)+anti-exon 17, 4=PBMC+PIBF-35 kDa (15 μg)+anti-exon 17. Binding to human lymphocytes and monocytes is suggestive for a PIBF receptor on these immune cells. A truncated version of rPIBF having exon 1-9 (48-kDa), having the N-terminus part of this functional PIBF forms and lacking the C-terminal part (exon 17-18) is not functional in terms of binding to immune cells (data not shown).

TABLE IV

| SEQ. ID. | | Tissues | Exon structure | Size of |
|---|---|---|---|---|
| Nos | Name/Definition | found | of mRNA (nt) | protein |
| No 1 | Published | | Exon1-18 | 758aa |
| | human full | | | 89-kDa |
| | length PIBF | | | |
| No 2, 3 | Alternative | Every cell | Exon 1-18 | 758aa |
| | human full | (nuclear) | | 89-kDa |
| | length PIBF | | | |
| No 4, 5 | mouse full | Every cell | Exon 1-18 | 757aa |
| | length PIBF | (nuclear) | | 89-kDa |
| No 6, 7 | Alternatively | Placenta | Exon (1-5)- | 298aa |
| | spliced | Lymphocytes | (17-18) | 35-kDa |
| | Human 35-kDa | Tumor (gastric | | |
| | | adenocc.) | | |
| No 8, 9 | Alternatively | Embryo | Exon (1-5)- | 297aa |
| | spliced mouse | placenta | (17-18) | 35-kDa |
| | 35-kDa (homologue | | | |
| | of No4) | | | |
| No | Alternatively | Tumor (endomet. | Exon 1-(13-18) | 87aa |
| 10, 11 | spliced human | adenocc) | | 10-kDa |
| 12, 13 | 10-kDa | Pregnancy lymphocytes | | |
| | | MCF-7 human mammary | Exon 1-(15-18) | |
| | | adenocc. | | |
| | | cell line | | |
| | | Leukocytes | Exon 14′-15-18 | |
| No 14 | Alternatively | Pregnancy lymphocytes | Exon 1-(9-10)- | 118aa |
| | spliced | MCF-7 tumor cell | (12-15)-(17-18) | 14-kDa |
| | human 14-kDa | line | | |
| No | Alternatively | Pregnancy lymphocytes | Exon 1-(9-10)- | 70aa |
| 15, 16 | spliced | MCF-7 tumor cell | (12-15)- | 8-kDa |
| | human 8-kDa | line | (17-18) | |
| No | Alternatively | MCF-7 tumor cell | Exon 1-(3-7)- | 185aa |
| 17, 18 | spliced | line | (9-10)-12-(17-18) | 20-kDa |
| | human 20-kDa | | | |
| No | Alternatively | MCF-7 tumor cell | Exon (1-7)-(9-15)- | 308aa |
| 19, 21, | spliced human | line | (17-18) | 37-kDa |
| 22 | 37-kDa | | Exon (1-7)-(9-10)- | |
| | | | 12-(17-18) | |
| No 20 | Alternatively | MCF-7 tumor cell | Exon (1-7)-(9-15)- | 258aa |
| | spliced | line | (17-18) | 31-kDa |
| | human 31-kDa | | | |
| No | Alternatively | Embryo | Exon (1-2)-(17-18) | 157aa |
| 23, 24 | spliced mouse | Placenta | | 18-kDa |
| | 18-kDa | | | |
| No | Alternatively | Placenta | Exon (1-4)-(16-18) | 256aa |
| 25, 26 | spliced | | | 30-kDa |
| | mouse 30-kDa | | | |
| No | Alternatively | Adult testis | Exon (1-2)-(15-18) | 229aa |
| 27, 28 | spliced mouse | | | 27-kDa |
| | 27-kDa | | | |
| No | Alternatively | Embryo | Exon (1-11)- | 512aa |
| 29, 30 | spliced mouse | | (17-18) | 61-kDa |
| | 61-kDa | | | |
| No | Alternatively | Embryo | Exon (1-11)-18 | 569aa |
| 31, 33 | spliced mouse | | | 68-kDa |
| | 68-kDa | | | |
| No 32 | Alternatively | Embryo | Exon (1-11)-18 | 86aa |
| | spliced mouse | | | 10.5-kDa |
| | 10.5-kDa | | | |
| No | Alternatively | Embryo | Exon 1-(8-14)- | 309aa |
| 34, 35 | spliced mouse | | (17-18) | 37-kDa |
| | 37-kDa | | | |
| No | Alternatively | Adult testis | Exon (1-11)- | 641aa |
| 36, 37 | spliced mouse | | (15-18) | 76-kDa |
| | 76-kDa | | | |

FIGS. 12A to 12Q show the alternatively processed PIBF proteins whereby the exons are schematically represented:

FIG. 12A shows mouse full length 89-kDA PIBF (SEQ ID No. 4), FIG. 12B shows exons (1-5)−(17-18) which is found in stomach tumor, human terminal placenta, male and female lymphocytes, female pregnancy lymphocytes (SEQ ID No. 6), FIG. 12C shows exons (1-5) −(17-18), found in mouse placenta and embryo (SEQ.ID No. 8), FIG. 12D shows exons 1−(13-18) (SEQ. ID No. 11), FIG. 12E shows exons 1−(15-18), found in MCF-7 cells and pregnancy lymphocytes (SEQ.ID No. 12), FIG. 12F shows a part of intron 14 and exons 15-18, found in leukocyte cDNA library (SEQ. ID No. 13), FIG. 12G shows exons 1+(9-10)+(12-15)+(17-18), found in MCF-7 cells and pregnancy lymphocytes (SEQ. ID No. 14), FIG. 12H shows exons 1+(3-7)+(9-10)+12+(17-18), found in MCF-7 cells—human mammary tumor cell line (SEQ. ID No. 17), FIG. 12I shows exon (1-7)+(9-15)+(17-18), found in MCF-7 cells—human mammary tumor cell line (SEQ. ID No. 19), FIG. 12J shows exon (1-7)+(9-10)+12+(17-18), found in MCF-7 cells—human mammary tumor cell line (SEQ. ID No. 22), FIG. 12K shows exon (1-2)−(17-18) found in mouse embryo and placenta (SEQ. ID No. 23), FIG. 12L shows exon (1-4)−(16-18) found in mouse placenta (SEQ. ID No. 25), FIG. 12M shows exons (1-2)−(15-18) found in mouse testis (SEQ. ID No. 27), FIG. 12N shows exon 1-11 18 found in mouse embryo (SEQ. ID No. 29), FIG. 120 shows exons (1-11)−18 found in mouse embryo (SEQ. ID No. 31), FIG. 12P shows exons 1−(8-14)−(17-18) found in mouse embryo (SEQ. ID No. 34) and FIG. 12Q shows exons (1-1)−(15-18) found in mouse test is (SEQ. ID No. 36).

### References

1. Szekeres-Bartho, J., Kinsky, R., Kapovic, M., Vargan P., Chaouat, G. Immuno-endocrine networks in pregnancy. In: Gergely et. al Ed. Progress in Immunology X. Springer Verlag 1993 p.861

2. Szekeres-Bartho, J., Reznikoff-Etievant, M. F., Varga, P., Varga, Z., Chaouat, G. Lymphocytic progesterone receptors in human pregnancy. J. Reprod Immunol. 16, 239, 1989.

3. Szekeres-Bartho, J., Szekeres, Gy., Debre, P., Autran, B., Chaouat, G. Reactivity of lymphocytes to a progesterone receptor-specific monoclonal antibody. Cell. Immunol. 125, 273, 1990.

4. Szekeres-Bartho, J., F. Kilar, G. Falkay, V. Csernus, A. Torok, and A. S. Pacsa. The mechanism of the inhibitory effect of progesterone on lymphocyte cytotoxicity: I. Progesterone-treated lymphocytes release a substance inhibiting cytotoxicity and prostaglandin synthesis. Am. J. Reprod. Immunol Microbiol. 9,15, 1985.

5. Szekeres-Bartho, J., Autran, B., Debre, P., Andreu, G., Denver, L., Chaouat, G. Immunoregulatory effects of a suppressor factor from healthy pregnant women's lymphocytes after progesterone induction. Cell. Immunol. 122, 281, 1989.

6. Szekeres-Bartho, J., Kinsky, R., Chaouat, G. A progesterone-induced immunologic blocking factor corrects high resorption rate in mice treated with antiprogesterone. Am. J. Ob. Gyn. 163, 1320, 1 990.

7. Szekeres-Bartho, J., Chaouat, G. Lymphocyte-derived progesterone induced blocking factor corrects resorption in a murine abortion system. Am. J. Reprod. Immunol. 23, 26, 1990.

8. Szekeres-Bartho, J., Faust, Zs., Varga, P. Progesterone induced blocking factor (PIBF) in normal and pathological pregnancy. Am. J. Reprod. Immunol. 34, 342, 1995.

9. Check, J., Arwitz, M., Gross, J., Szekeres-Bartho, J., Chung, H. Wu. Evidence that the expression of a progesterone-induced blocking factor by maternal T-lymphocytes is positively correlated with conception. Am. J. Reprod. Immunol 38, 6, 1997.

10. Szekeres-Bartho, J., Kinsky, R., Chaouat, G. The effect of a progesterone induced immunologic blocking factor on NK-mediated resorption. Am. J. Reprod. Immunol. 24, 105. 1990.

11. Szekeres-Bartho, J., G. Par, Gy. Dombay, Smart, Y. C. Z. Volgyi. The anti-abortive effect of PIBF in mice is manifested by modulating NK activity. Cell. Immunol. 177, 194, 1997.

12. Faust, Zs. G. Laskarin˜D. Rukavina, J. Szekeres-Bartho Progesterone Induced Blocking Factor Inhibits Degranulation of NK Cells Am. J. Reprod. Immunol. 42, 71, 1999.

13. Szekeres-Bartho, J., Wegmann, T. G., A progesterne-dependent immunomodulatory protein alters the Th1/Th2 balanced. Reprod. Immunol. 31, 81-95, 1996.

14. Szekeres-Bartho, J., Wegmann, T. G. Kelemen, K, Bognar, I., Faust, Zs., Varga, P. Interaction of progesterone- and cytokine-mediated immunomodulatory mechanisms in favor of successful gestation Regional Immunolo˜v, 6, 315, 1995.

15. Szekeres-Bartho, J., Faust, Zs., Varga, P., Szereday, L., Kelemen, K. The immunological pregnancy protective effect of progesterone is manifested via controlling cytokine production. Am. J. Reprod. Immunol. 35, 348, 1996.

16. Kelemen, K., Bognar, 1., Paal, M., and Szekeres-Bartho, J. A progesterone-induced protein increases the synthesis of asymmetric antibodies. Cell. Immunol. 167, 129, 1996.

17. Algarra I, Collado A, Garrido F. Altered MHC class I antigens in tumors. Int. J. Clin. Lal7. Res. 27,95, 1997.

18. Rees R C, Mian S. Selective MHC expression in tumors modulates adaptive and innate antitumor responses. Cancer Immunol. Immunother. 48, 374, 1999.

19. Whiteside, T. L., and Herberman, R. B. The role of natural killer cells in immune surveillance of cancer. cu7′r. Opin. Immunol. 7. 704. 1995.

20. Karlhofer F M, Ribaudo R K, and Yokoyama W M. MHC class I alloantigen specificity of Ly-49+ IL-2activated natural killer cells. Nahlre. 358. pp.66-70. 1992.

21. Phillips J H, Gumperz J E, Parham P. and Lanier L L. Superantigen-dependent, cell-mediated cytotoxicity inhibited by MHC class I receptors on T Iymphocytes. Science 268. pp.403-405. 1995.

22. Cabestre F A, Lefebvre S. Moreau P. Rouas-Friess N. Dausset J. Carosella E D, Paul P. HLA-G expression: immune privilege for tumour cells?Semin. Cancer Biol. 9(1)pp.27-36. 1999.

23. Kagi D, Lederman B: Burki K et al. Cytotoxicity mediated by T cells and natural killer cells is greatly impaired in perforin-deficient mice. Nature 369. pp.31-37. 1994.

24. Smyth, M. J. et al. Perforin is a major contributor to NK cell control of tumor metastasis. J. Immunol. 162, 6658, 1999.

25. Kollias G. Douni E, Kassiotis G. Kontoyiannis D. On the role of tumor necrosis factor and receptors in models of multiorgan failure, rheumatoid arthritis, multiple sclerosis and inflammatory bowel disease Imm unol Rev. 169, 175, 1999

26. Manilay J O, Sykes M. Natural killer cells and their role in graft rejection. Cu7r Op7n Immunol. 10, 532,1998.

27. Young N T. Kir genes, killer cells and clinical transplantation. Transplantation 68, 1626, 1999

28. Beaman K, Angkachatchai V, Gilman-Sachs A. T J6: the pregnancy-associated cytokine. Am J Reprod Immunol. 35, 338, 1996.

29. Aslakson C J, Lee G. Boomer J S, Gilman-Sachs A, Kucuk O. Beaman K D. Expression of regeneration and tolerance factor on B cell chronic lymphocytic leukemias: a possible mechanism for escaping immune surveillance. Am JHematol 61, 46, 199.

30. Bonavida B. Bradley T P' Grimm E A. Frequency determination of killer cells by a single-cell cytotoxic assay. Methods Enzymol 93, 270, 1983.

We claim:

1. A method for diagnosing a tumor in a patient said method comprising taking a sample from the patient, measuring the concentration of PIBF (Progesterone Induced Blocking Factor) or a derivative thereof or a fragment thereof in the sample and determining whether the concentration of PIBF in the sample is above or below a predetermined threshold value, whereby the concentration above the threshold value identifies a patient with a tumor.

2. The method according to claim 1 characterized in that the tumor is an epithelial carcinoma, especially a lung carcinoma, a colon carcinoma or a breast carcinoma.

3. The method according to claim 1 or 2 characterized in that the sample is a body fluid, especially urine or serum.

4. The method according to claim 1 or 2 characterized in that the sample is a tissue sample.

5. The method according to any one of claims 1 or 2 characterized in that the threshold value is the concentration of PIBF in a sample of a healthy person.

6. The method according to any one of claims 1 or 2 characterized in that the threshold value is determined by measuring the concentration of PIBF in at least one healthy person parallel to the determination of the concentration of PIBF in a sample of the patient.

7. The method according to any one of claims 1 or 2 characterized in that as a positive control the concentration of PIBF or a derivative thereof or a fragment thereof in the sample comprising a defined concentration of PIBF or a derivative thereof or a fragment thereof is measured parallel to the determination of the concentration of PIBF in the sample of the patient.

8. The method according to any one of claims 1 or 2 characterized in that the concentration of PIBF in the sample is measured immunologically, in particular by a competitive assay, by a sandwich assay, by immunostaining or combinations of these methods.

9. The method according to any one of claims 1 or 2 characterized in that the concentration of PIBF in the sample is measured indirectly by measuring the concentration of PIBF-mRNA in the sample.

10. A method for determining the positive or negative progression of a tumor in a patient comprising diagnosing a tumor in a patient according to a method according to any one of claims 1 or 2 and determining whether the measured concentration of PIBF or a derivative thereof or a fragment thereof in the sample is above or below at least one previously measured concentration of PIBF or a derivative thereof or a fragment thereof in at least one sample previously taken from the same patient, whereby the concentration above the previously measured concentration identifies a positive progression.

11. Use of an anti-PIBF antibody, preferably a monoclonal antibody, or a fragment thereof in a method according to claim 8.

12. Use of PIBF or a derivative thereof or a fragment thereof in a method according to claim 8.

13. The use according to claim 12 characterized in that the PIBF is a recombinant PIBF.

14. A kit comprising a first reagent comprising at least one anti-PIBF antibody or a fragment thereof and a second reagent comprising PIBF or a derivative thereof or a fragment thereof at a defined concentration.

15. The kit according to claim 14 characterized in that it comprises a solid phase to which the at least one anti-PIBF antibody or the fragment thereof or the PIBF or the derivative thereof or the fragment thereof is bound.

16. The kit according to claim 14 or 15 characterized in that the PIBF is recombinant.

17. The kit according to claim 14 or 15 characterized in that it comprises a further reagent comprising a second anti-PIBF antibody or a fragment thereof which binds to an epitope of the PIBF which is distinct to the epitope recognized by the first anti-PIBF antibody or the fragment thereof.

18. The kit according to any one of claims 14 or 15 for diagnosing a tumor in a patient and for determining the progression of a tumor in a patient, respectively.

19. Use of an anti-PIBF antibody, preferably a monoclonal anti PIBF antibody, especially humanized anti-PIBF antibody, or a fragment thereof for the preparation of an anti-tumor medicine.

20. The use according to claim 19 characterized in that the antibody is a single chain antibody.

21. The use according to claim 19 or 20 characterized in that the antibody has attached thereto a molecule.

22. The use according to claim 21 characterized in that the molecule is a toxic substance, especially a radionuclide a chemotherapeutic drug or a toxin, and a prodrug, respectively.

23. Use of PIBF or a derivative thereof or a fragment thereof for the preparation of an anti-tumor medicine.

24. The use according to claim 23 characterized in that the medicine is a vaccine.

25. The use according to claim 24 characterized in that the vaccine further comprises an adjuvant.

26. The use according to claim 23 or 24 characterized in that the PIBF or the derivative thereof or the fragment thereof is recombinant.

27. The use according to claim 23 to 24 characterized in that the PIBF or the derivative or the fragment thereof is a chemically synthesized molecule.

28. The use of a polynucleotide encoding PIBF or a derivative thereof or a fragment thereof or PIBF-antisense molecule for the preparation of an anti-tumor medicine.

29. Recombinant protein with a Progesterone-Induced Immunomodulatory Protein- (PIBF-) activity, comprising (i) the amino acid sequence according to SEQ. ID. NO 1 or (ii) an amino acid sequence with an amino acid identity of at least 98% to the sequence according to SEQ. ID. NO 1 as determined by FAST/A algorithm or (iii) an amino acid sequence with an amino acid identity of at least 95% to the sequence from amino acid residue 580 to 630 of SEQ. ID. NO 1 as determined by FAST/A algorithm and having a PIBF activity of at least 50% of the natural human PIBF molecule.

30. Recombinant protein according to claim 29, characterized in that it comprises an amino acid sequence as given from amino acid residues 300 to 350 in SEQ. ID. NO 1.

31. Recombinant protein according to claim 29 or 30, characterized in that it comprises an amino acid sequence as given from amino acid residues 580 to 630 in SEQ. ID. NO 1.

32. Protein with a PIBF activity comprising—the amino acid sequence according to SEQ. ID. NO 4 or -an amino acid with an amino acid identity of at least 90%, preferably at least 95%, still preferred at least 99%, to the sequence according to SEQ. ID. No 4 as de termined by FAST/A algorithm.

33. Protein characterized in that it comprises an amino acid sequence with an identity of at least 85%, preferably at least 90%, still preferred at least 95% as determined by FAST/A algorithm to a sequence selected from the group consisting of SEQ. ID. NOs 6, 8, 10, 14, 15, 17, 19, 20, 23, 25, 27, 29, 31, 32, 34 and 36 said protein being an alternatively processed PIBF protein.

34. Nucleic acid molecule encoding a recombinant protein with a PIBF activity according to any one of claims 29, 30, or 32.

35. Nucleic acid molecule encoding an alternatively processed PIBF protein characterized in that it comprises a nucleic acid sequence with an identity of at least 80%, preferably at least 90%, still preferred at least 95% to -a sequence elected from the group consisting of SEQ. ID. NOs 7, 9, 11, 12, 13, 16, 18, 21, 22, 24, 26, 28, 30, 33, 35 and 37 or -a sequence which hybridizes under stringent conditions to one of the above sequences or -a sequence which is degenerated due to the genetic code to one of the above sequences.

36. Nucleic acid vector comprising a nucleic acid sequence according to claim 34 and a suitable regulatory element.

37. Nucleic acid vector comprising a nucleic acid sequence according to claim 35 and a suitable regulatory element.

38. Nucleic acid vector according to claim 36, characterized in that it further comprises a selection marker.

39. Nucleic acid vector according to claim 37, characterized in that it further comprises a selection marker.

41. Cell, comprising a vector according to claim 37 or 39.

42. Cell, comprising a vector according to claim 38.


SEQUENCE LISTING (37 sequences)

SEQ ID NO: 1 - protein, 757 aa, Homo sapiens
MSRKISKESKKVNISSSLESEDISLETTVPTDDISSSEEREGKVRITRQLIERKELLHNIQLLKIELSQKTMMIDNLKVDYLTKIEELEEKLNDALHQKQLLTLRLDNQLAFQQKDASKYQELMKQEMETILLRQKQLEETNLQLREKAGDVRRSLRDFELTEEQYIKLKAFPEDQLSIPEYVSVRFYELVNPLRKEICELQVKKNILAEELSTNKNQLKQLTETYEEDRKNYSEVQIRCQRLALELADTKQLIQQGDYRQENYDKVKSERDALEQEVIELRRKHEILEASHMIQTKERSELSKEVVTLEQTVTLLQKDKEYLNRQNMELSVRCAHEEDRLERLQAQLEESKKAREEMYEKYVASRDHYKTEYENKLHDELEQIRLKTNQEIDQLRNASREMYERENRNLREARDNAVAEKERAVMAEKDALEKHDQLLDRYRELQLSTESKVTEFLHQSKLKSFESERVQLLQEETARNLTQCQLECEKYQKKLEVLTKEFYSLQASSEKRITELQAQNSEHQARLDIYEKLEKELDEIIMQTAEIENEDEAERVLFSYGYGANVPTTAKRRLKQSVHLARRVLQLEKQNSLILKDLEHRKDQVTQLSQELDRANSLLNQTQQPYRYLIESVRQRDSKIDSLTESIAQLEKDVSNLNKEKSALLQTKNQMALDLEQLLNHREELAAMKQILVKMHSKHSENSLLLTKTEPKHVTENQKSKTLNVPKEHEDNIFTPKPTLFTKKEAPEWSKKQKMKT

SEQ ID NO: 2 - protein, 758 aa, Homo sapiens
  UNSURE 595: Xaa = Any amino acid
MSRKISKESKKVNISSSLESEDISLETTVPTDDISSSEEREGKVRITRQLIERKELLHNIQLLKIELSQKTMMIDNLKVDYLTKIEELEEKLNDALHQKQLLTLRLDNQLAFQQKDASKYQELMKQEMETILLRQKQLEETNLQLREKAGDVRRSLRDFELTEEQYIKLKAFPEDQLSIPEYVSVRFYELVNPLRKEICELQVKKNILAEELSTNKNQLKQLTETYEEDRKNYSEVQIRCQRLALELADTKQLIQQGDYRQENYDKVKSERDALEQEVIELRRKHEILEASHMIQTKERSELSKEVVTLEQTVTLLQKDKEYLNRQNMELSVCCAHEEDRLERLQAQLEESKKAREEMYEKYVASRDHYKTEYENKLHDELEQIRLKTNQEIDQLRNASREMYERENRNLREARDNAVAEKERAVMAEKDALEKHDQLLDRYRELQLSTESKVTEFLHQSKLKSFESERVQLLQEETARNLTQCQLECEKYQKKLEVLTKEFYSLQASSEKRITELQAQNSEHQARLDIYEKLEKELDEIIMQTAEIENEDEAERVLFSYGYGANVPTTAKRRLKQSVHLARRVLQLEKQNSLIXKRSGTSKGPSNTAFTRSLTEANSLLNQTQQPYRYLIESVRQRDSKIDSLTESIAQLEKDVSNLNKEKSALLQTKNQMALDLEQLLNHREELAAMKQILVKMHSKHSENSLLLTKTEPKHVTENQKSKTLNVPKEHEDNIFTPKPTLFTKKEAPEWSKKQKMKT

SEQ ID NO: 3 - DNA, 2715 bp, Homo sapiens
cgtgttttgctttccgctcctcggaacatccgggagagttgacttccggcggcttgtgggagtgctggttctgtcctccttgcgggtgcggagatggttgtcttggttacgggtcctaacggtcccctgccttgaaatcccttgttgagggcctgcaaccttgtgcttccgactggagacgcctttggtccctcggtgtctgcactggctgctggtcaaggcttcagtgtggacgaattgacactttcgagaatattaaaatcaaattagagaagaaaactgatccataataataaaaatgtctcgaaaaatttcaaaggagtcaaaaaaagtgaacatctctagttctctggaatctgaagatattagtttagaaacaacagttcctacggatgatatttcctcatcagaagagcgagagggcaaagtcagaatcaccaggcagctaattgaacgaaaagaactacttcataatattcagttactaaaaattgagctatcccagaaaactatgatgatcgacaatttgaaagtggattatcttacaaagattgaagaattggaggagaaacttaatgatgcacttcaccagaagcagctactaacattgagattagacaaccaattggcttttcaacagaaagatgccagcaaatatcaagaattaatgaaacaagaaatggaaaccattttgttgagacagaaacaactagaagagacaaatcttcagctaagagaaaaagctggagatgttcgtcgaagcctgcgtgactttgagttgacagaagagcaatatattaaattaaaagcttttcctgaagatcagctttctattcctgaatatgtatctgttcgcttctatgagctagtgaatccattaagaaaggaaatctgtgaactacaagtgaaaaagaatatcctagcagaagaattaagtacaaacaaaaaccaactgaagcagctgacagagacatatgaggaagatcgaaaaaactactctgaagttcaaattagatgtcaacgtttggccttagaattagcagacacaaaacagttaattcagcaaggtgactaccgtcaagagaactatgataaagtcaagagtgaacgtgatgcacttgaacaggaagtaattgagcttaggagaaaacatgaaatacttgaagcctctcacatgattcaaacaaaagaacgaagtgaattatcaaaagaggtagtcaccttagagcaaactgttactttactgcaaaaggataaagaatatcttaatcgccaaaacatggagcttagtgttcgctgtgctcatgaagaggatcgccttgaaagacttcaagctcaactggaagaaagcaaaaaggctagagaagagatgtatgaaaaatatgtagcatccagagaccattataaaacagaatatgaaaataaactacatgatgaactagaacaaatcagattgaaaaccaaccaagaaattgatcaacttcgaaatgcctctagggaaatgtatgaacgagaaaacagaaatctccgagaagcaagggataatgctgtggctgaaaaggaacgagcagtgatggctgaaaaggatgctttagaaaaacacgatcagctcttagacaggtacagagaactacaacttagtacagaaagcaaagtaacagaatttctccatcaaagtaaattaaaatcttttgaaagtgagcgtgttcaacttctgcaagaggaaacagcaagaaatctcacacagtgtcaattggaatgtgaaaaatatcagaaaaaattggaggttttaaccaaagaattttatagtctccaagcctcttctgaaaaacgcattactgaacttcaagcacagaactcagagcatcaagcaaggctagacatttatgagaaactggaaaaagagcttgatgaaataataatgcaaactgcagaaattgaaaatgaagatgaggctgaaagggttcttttttcctacggctatggtgctaatgttcccacaacagccaaaagacgactaaagcaaagtgttcacttggcaagaagagtgcttcaattagaaaaacaaaactcgctgattttaaaagatctggaacatcgaaaggaccaagtaacacagctttcacaagagcttgacagagccaattcgctattaaaccagactcaacagccttacaggtatctcattgaatcagtgcgtcagagagattctaagattgattcactgacggaatctattgcacaacttgagaaagatgtcagcaacttaaataaagaaaagtcagctttactacagacgaagaatcaaatggcattagatttagaacaacttctaaatcatcgtgaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatggaaaacaaaattcagcttaatcgtgtactcagcattttttaaataacaatgtttatttgaactaatattaaattaacaaattcg

SEQ ID NO: 4 - protein, 756 aa, Mus musculus
MSRKIAKEPKKVNISSSLESEDISLETTIHTDDVSSSEEREGKVKITRQLIERKEILHNIQLLKIELSQKNMMIDNLKMDYLTKIEELEEKLNDALHQKQLLTLRLDNQLTIQQKDAKKYQELMKQEMETILLRQKQLEETNHQLREKAGDVRRNLRDFELTEEQYVKLKSFPEDQLSIPEYVSIRFYELVNPLRKEVCELQVKKSELSEELSTSKGQLKQLTETYEEDRRNNAELLIRCQRLTLELADTKQLVQQGDYRQENYDKVKSERDALEQDVLELRRKHEVLEASHIAQAKERNELSKEVSSLQQTVTLLQKDKDYLNRQNMELSVRCAHEEDRLERLQVQLEDTKKAREEMYEKYVTSRDHYKTEYENKLHDELEQIKLKTNLEIDQLRSASREMYERENRNLREARDNALAEKNRAVAAEKDALGKHEQLLDRYRELQLSTESKVSEFLHQSKLKSFESERVQLLQEETARNLTQCQLECEKYQKKLEVLTKEFYSLQTSSEKRITELEAQNSEHQARLDIYEKLEKELDEIIMQTAEIENEDEAERILYSYGYGANVPTTAKRRLKQSVHLARRVLQLEKQNSLILKDLDHQKNQVRQLSQELDRANSLLNQTQQPYRYLIESVRQRDAKIDSLMKSTAQLEKDVSNLNKEKSALLQTKNQMALDLEQLLSHREEFAAMKQIIINMCSKHSENNLFLTKMESKSVTENQAKTLNMPREHEENIFIPKPTLFTKKEAQEWPKSQKMKT

SEQ ID NO: 5 - DNA, 2719 bp, Mus musculus
acggccagtgaattgtaatacgactcactatagggcgaattgggccctctagatgcatgctcgagcggccgccagtgtgatggatatctgcagaattcgcccttggttgtcttggttacgggtcctaacggtccggtagccccgagatacctgttgaggggtggcagcctgagctgctgactgaagacgccattggttcttccaagagtccgggtgaagctggtgttatccttatatgcggagttactgaccattgagagaagattgattcaaataataaaatgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagattgaggagctagaggaaaaacttaatgacgcccttcaccagaagcagctgctaactttgcgattagacaatcagttgactattcaacagaaagatgccaaaaaatatcaagaactaatgaaacaagaaatggaaaccattttattgcgacagaagcaactggaagaaacaaaccatcagctgagagaaaaggctggagatgttcgccgaaatctgcgagactttgagctgacagaagagcagtatgtgaagctaaaatcttttcctgaagatcaactctctattcctgaatatgtatctattcgcttctatgagctcgtgaacccattaagaaaggaagtctgtgagctacaggtgaagaagagtgaactctctgaagaactgagtacaagtaaaggccaactgaagcagctgacggagacatatgaagaagatcgaagaaacaacgctgaacttctaattcgatgtcaacgtttgaccttagaattagcagacacaaaacagttagttcagcaaggtgattaccgtcaagagaactatgacaaagtgaagagtgaacgcgatgctctggaacaggacgtactcgagcttagaagaaaacacgaagtacttgaagcctctcacatagctcaagctaaggaaaggaatgaattatcaaaggaggtcagcagcctgcagcagacagtcaccctgctgcagaaggataaagactacctcaatcgccaaaacatggaactcagtgtacgctgtgcccatgaggaggatcggctggaaaggctgcaagttcaactggaagacaccaaaaaggctagagaagagatgtatgagaaatatgtcacgtccagagaccattataaaacagaatatgaaaataaactacatgatgaactggaacaaatcaaattgaaaactaatctagaaattgatcagcttcgaagtgcctctagggaaatgtatgaacgagaaaacagaaatctccgtgaagcaagggataatgcactcgctgaaaagaaccgagcagtggcagcggaaaaggacgctctgggaaagcatgagcagctcctagacaggtacagagaactccagctcagtacagagagcaaggtatctgagtttctccatcagagcaagttgaagtcctttgaaagtgagcgtgttcaactcctgcaagaggaaactgcaagaaatctcacgcagtgccagttggagtgtgaaaaatatcagaagaaattggaggttttaactaaagaattttatagtctccaaacttcttctgaaaaacgcattaccgaactcgaggcacagaactcagagcatcaggcaaggttagacatttacgagaagttggaaaaggagcttgatgagataataatgcaaacagcagaaattgaaaatgaagatgaggctgaaagaattctttattcctatggttatggtgctaatgttcccacaacagctaaaagacgactaaagcaaagtgtccacttggcaagaagagttcttcagttagaaaaacaaaattcattaattttaaaagatctggaccatcaaaagaaccaagtaagacagctttcacaagagcttgacagagccaattcactgttgaatcagactcagcagccctacagatacctcatagagtctgtgcgacagagagatgccaagattgactcactgatgaagtctacagctcaacttgagaaagatgtcagcaacttaaataaagagaagtcagccctgctgcagacgaagaaccagatggcactggatctggagcagctcctcagtcaccgcgaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcagaagatgaagacctagtgtttggtgttgaagaaagctggagcctaacccagccaggccctcggctctccattggaacaggcctgtgttatcatgtactcctgaagtgcaatgtctgttggaagggcgaattccagcacactggcggccgttactagtggatccgagctcggtaccaagctggc

SEQ ID NO: 6 - protein, 298 aa, Homo sapiens
MSRKISKESKKVNISSSLESEDISLETTVPTDDISSSEEREGKVRITRQLIERKELLHNIQLLKIELSQKTMMIDNLKVDYLTKIEELEEKLNDALHQKQLLTLRLDNQLAFQQKDASKYQELMKQEMETILLRQKQLEETNLQLREKAGDVRRNLRDFELTEEQYIKLKAFPEDQLSIPEYVSVRFYELVNPLRKEICELQVKKNILAEELSTNKNQLKQLTEELAAMKQILVKMHSKHSENSLLLTKTEPKHVTENQKSKTLNVPREHEDNIFTPKPTLFTKKEAPEWSKKQKMKT

SEQ ID NO: 7 - DNA, 957 bp, Homo sapiens
atgtctcgaaaaatttcaaaggagtcaaaaaaagtgaacatctctagttctctggaatctgaagatattagtttagaaacaacagttcctacggatgatatttcctcatcagaagagcgagagggcaaagtcagaatcaccaggcagctaattgaacgaaaagaactacttcataatattcagttactaaaaattgagctatcccagaaaactatgatgatcgacaatttgaaagtggattatcttacaaagattgaagaattggaggagaaacttaatgatgcacttcaccagaagcagctactaacattgagattagacaaccaattggcttttcaacagaaagatgccagcaaatatcaagaattaatgaaacaagaaatggaaaccattttgttgagacagaaacaactagaagagacaaatcttcagctaagagaaaaagctggagatgttcgtcgaaacctgcgtgactttgagttgacagaagagcaatatattaaattaaaagcttttcctgaagatcagctttctattcctgaatatgtatctgttcgcttctatgagctagtgaatccattaagaaaggaaatctgtgaactacaagtgaaaaagaatatcctagcagaagaattaagtacaaacaaaaaccaactgaagcagctgacagaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatgg

SEQ ID NO: 8 - protein, 297 aa, Mus musculus
MSRKIAKEPKKVNISSSLESEDISLETTIHTDDVSSSEEREGKVKITRQLIERKEILHNIQLLKIELSQKNMMIDNLKMDYLTKIEELEEKLNDALHQKQLLTLRLDNQLTIQQKDAKKYQELMKQEMETILLRQKQLEETNHQLREKAGDVRRNLRDFELTEEQYVKLKSFPEDQLSIPEYVSIRFYELVNPLRKEVCELQVKKSELSEELSTSKGQLKQLTEEFAAMKQIIINMCSKHSENNLFLTKMESKSVTENQAKTLNMPREHEENIFIPKPTLFTKKEAQEWPKSQKMKT

SEQ ID NO: 9 - DNA, 1173 bp, Mus musculus
gacggccgtgaattgtaatacgactcactatagggcgaattgggccctctagatgcatgctcgagcggccgccagtgtgatggatatctgcagaattcgcccttatgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagattgaggagctagaggaaaaacttaatgacgcccttcaccagaagcagctgctaactttgcgattagacaatcagttgactattcaacagaaagatgccaaaaaatatcaagaactaatgaaacaagaaatggaaaccattttattgcgacagaagcaactggaagaaacaaaccatcagctgagagaaaaggctggagatgttcgccgaaatctgcgagactttgagctgacagaagagcagtatgtgaagctaaaatcttttcctgaagatcaactctctattcctgaatatgtatctattcgcttctatgagctcgtgaacccattaagaaaggaagtctgtgagctacaggtgaagaagagtgaactctctgaagaactgagtacaagtaaaggccaactgaagcagctgacggaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcagaagatgaagacctagtgtttggtgttgaagaaagctggagcctaacccagccaggccctcggctctccattggaacaggcctgtgttatcatgtactcctgaagtgcaatgtctgttggaagggcgaattccagcacactggcggccgttactagtggatccgagctcggtaccaagcttggcgtaatca

SEQ ID NO: 10 - protein, 87 aa, Homo sapiens
MALDLEQLLNHREELAAMKQILVKMHSKHSENSLLLTKTEPKHVTENQKSKTLNVPKEHEDNIFTPKPTLFTKKEAPEWSKKQKMKT

SEQ ID NO: 11 - DNA, 983 bp, Homo sapiens
acgccagcttggtaccgagctcggatccactagtaacggccgccagtgtgctggaattcgcccttctgccttgaaatcccttgttgagggcctgcaaccttgtgcttccgactggagacgcctttggtccctcggtgtctgcactggctgctggtcaaggcttcagtgtggacgaattgacactttcgagttgaaaatgaagatgaggctgaaagggttcttttttcctacggctatggtgctaatgttcccacaacagccaaaagacgactaaagcaaagtgttcacttggcaagaagagtgcttcaattagaaaaacaaaactcgctgattttaaaagatctggaacatcgaaaggaccaagtaacacagctttcacaagagcttgacagagccaattcgctattaaaccagactcaacagccttacaggtatctcattgaatcagtgcgtcagagagattctaagattgattcactgacggaatctattgcacaacttgagaaagatgtcagcaacttaaataaagaaaagtcagctttactacagacgaagaatcaaatggcattagatttagaacaacttctaaatcatcgtgaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatggaagggcgaattctgcagatatccatcacactggcggccgctcgagcatgcatctagagggcccaattcgccctatagtgagtcgtattacaattcact

SEQ ID NO: 12 - DNA, 689 bp, Homo sapiens
ggttgtcttggttacgggtcctaacggtcccctgccttgaaatcccttgttgagggcctgcaaccttgtgcttccgactggagacgcctttggtccctcggtgtctgcactggctgctggtcaaggcttcagtgtggagtaattgacactttcgagcttgacagagccaattcgctattaaaccagactcaacagccttacaggtatctcattgaatcagtgcgtcagagagattctaagattgattcactgacggaatctattgcacaacttgagaaagatgtcagcaacttaaataaagaaaagtcagctttactacagacgaagaatcaaatggcattagatttagaacaacttctaaatcatcgtgaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatggaaaacaaaattcagcttaatcgtgtactcagc

SEQ ID NO: 13 - DNA, 1257 bp, Homo sapiens
ccatcagaaaaagaacttcaggacgcgtgggtttatcgcccagactggagtgcagtggcatgatcacagttcattgcaacctctgcctcccagggtcaaggactcctccctcctcagcctcccaagtagctggaactgtagctacgcactactgtgcctggctaatttttgtatttttttggtagagacagggtttcaccatgttgcccaggctagtctagaacttctgggctcaagcgatccacctgcctagggcctctgaaagtactgggattggagatgtgccactgcacccagccaagaagttaatattttaaaagttttaaaaactatttctcttataacaaagggttttttcaagtcatacattaaataacattaatatatgttgtttattatttgttttcttaaggatctgtttatattctttagagtgtcttttcatactataacattaggaggatctttatcctcaaatttcgaaagaccagaaatacttcattttattgcagttcctgacacatagttaatgcttggggttgacacagtggtgtgttggtaaatgttaacagccagctttccaagagggatatgagggagagcttgatttgtaacattggcttgtatctcttttataaatactccccaccatggctgacttcaaactaccaacctaaggttactgaagatggagtaaagattgtcagcagcataccagtacttaatgtttgcaccatacagatacaacagacatagcttgacagagccaattcgctattaaaccagactcaacagccttacaggtatctcattgaatcagtgcgtcagagagattctaagattgattcactgacggaatctattgcacaacttgagaaagatgtcagcaacttaaataaagaaaagtcagctttactacagacgaagaatcaaatggcattagatttagaacaacttctaaatcatcgtgaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatgg

SEQ ID NO: 14 - protein, 118 aa, Homo sapiens
MQTAEIENEDEAERVLFSYGYGANVPTTAKRRLKQSVHLARRVLQLEKQNSLILKDLEHRKDQVTQLSQELDRANSLLNQTQQPYRYLIESVRQRDSKIDSLTESIAQLEKDVRNWQQ

SEQ ID NO: 15 - protein, 70 aa, Homo sapiens
MKQILVKMHSKHSENSLLLTKTEPKHVTENQKSKTLNVPKEHEDNIFTPKPTLFTKKEAPEWSKKQEMKT

SEQ ID NO: 16 - DNA, 1173 bp, Homo sapiens
ggttgtcttggttacgggtcctaacggtcccctgccttgaaatcccttgttgagggcctgcaaccttgtgcttccgactggagacgcctttggtccctcggtgtctgcactggctgctggtcaaggcttcagtgtggagtaattgacactttcgagagaccattataaaacagaatatgaaaataaactacatgatgaactagaacaaatcagattgaaaaccaaccaagaaattgatcaacttcgaaatgcctctagggaaatgtatgaacgagaaaacagaaatctccgagaagcaagggataatgctgtggctgaaaaggaacgagcagtgatggctgaaaaggatgctttagaaaaacacgatcagctcttagacaggttttaaccaaagaattttatagtctccaagcctcttctgaaaaacgcattactgaacttcaagcacagaactcagagcatcaagcaaggctagacatttatgagaaactggaaaaagagcttgatgaaataataatgcaaactgcagaaattgaaaatgaagatgaggctgaaagggttcttttttcctacggctatggtgctaatgttcccacaacagccaaaagacgactaaagcaaagtgttcacttggcaagaagagtgcttcaattagaaaaacaaaactcgctgattttaaaagatctggaacatcgaaaggaccaagtaacacagctttcacaagagcttgacagagccaattcgctattaaaccagactcaacagccttacaggtatctcattgaatcagtgcgtcagagagattctaagattgattcactgacggaatctattgcacaacttgagaaagatgtcaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaagagatgaagacctagtgttttggatgggaagcacctgtagaccattatatactcctgaagttctttttctgatggaaaacaaaattcagcttaatcgtgtactcac

SEQ ID NO: 17 - protein, 185 aa, Homo sapiens
MKQEMETILLRQKQLEETNLQLREKAGDVRRNLRDFELTEEQYIKLKAFPEDQLSIPEYVSVRFYELVNPLRKEICELQVKKNILAEELSTNKNQLKQLTETYEEDRKNYSEVQIRCQRLALELADTKQLIQQGDYRQENYDKVKSERDALEQEVIELRRKHEILEASHMIQTKERSELSKERPL

SEQ ID NO: 18 - DNA, 1596 bp, Homo sapiens
ccgagctcggatccactagtaacggccgccagtgtgctggaattcgcccttctgccttgaaatcccttgttgagggcctgcaaccttgtgcttccgactggagacgcctttggtccctcggtgtctgcactggctgctggtcaaggcttcagtgtggagtaattgacactttcgagattgaagaattggaggagaaacttaatgatgcacttcaccagaagcagctactaacattgagattagacaaccaattggcttttcaacagaaagatgccagcaaatatcaagaattaatgaaacaagaaatggaaaccattttgttgagacagaaacaactagaagagacaaatcttcagctaagagaaaaagctggagatgttcgtcgaaacctgcgtgactttgagttgacagaagagcaatatattaaattaaaagcttttcctgaagatcagctttctattcctgaatatgtatctgttcgcttctatgagctagtgaatccattaagaaaggaaatctgtgaactacaagtgaaaaagaatatcctagcagaagaattaagtacaaacaaaaaccaactgaagcagctgacagagacatatgaggaagatcgaaaaaactactctgaagttcaaattagatgtcaacgtttggccttagaattagcagacacaaaacagttaattcagcaaggtgactaccgtcaagagaactatgataaagtcaagagtgaacgtgatgcacttgaacaggaagtaattgagcttaggagaaaacatgaaatacttgaagcctctcacatgattcaaacaaaagaacgaagtgaattatcaaaagagagaccattataaaacagaatatgaaaataaactacatgatgaactagaacaaatcagattgaaaaccaaccaagaaattgatcaacttcgaaatgcctctagggaaatgtatgaacgagaaaacagaaatctccgagaagcaagggataatgctgtggctgaaaaggaacgagcagtgatggctgaaaaggatgctttagaaaaacacgatcagctcttagacaggttttaaccaaagaattttatagtctccaagcctcttctgaaaaacgcattactgaacttcaagcacagaactcagagcatcaagcaaggctagacatttatgagaaactggaaaaagagcttgatgaaataataatgcaaactgcagaaagaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatggaagggcgaattctgcagatatccatcacactggcggccgctcgagcatgcatctagagggcccaattcgccctatagtgagtcgtattacaattca

SEQ ID NO: 19 - protein, 308 aa, Homo sapiens [sequence omitted]

SEQ ID NO: 20 - protein, 258 aa, Homo sapiens
MYERENRNLREARDNAVAEKERAVMAEKDALEKHDQLLDRYRELQLSTESKVTEFLHQSKLKSFESERVQLLQEETARNLTQCQLECEKYQKKLEVLTKEFYSLQASSEKRITELQAQNSEHQARLDIYEKLEKELDEIIMQTAEIENEDEAERVLFSYGYGANVPTTAKRRLKQSVHLARRVLQLEKQNSLILKDLEHRKDQVTQLSQELDRANSLLNQTQQPYRYLIESVRQRDSKIDSLTESIAQLEKDVRNWQQ

SEQ ID NO: 21 - DNA, 2403 bp, Homo sapiens
gcttggtaccgagctcggatccactagtaacggccgccagtgtgctggaattcgcccttcgccttgaaatcccttgttgagggcctgcaaccttgtgcttccgactggagacgcctttggtccctcggtgtctgcactggctgctggtcaaggcttcagtgtggagtaattgacactttcgagaatattaaaatcaaattagagaagaaaactgatccataataataaaaatgtctcgaaaaatttcaaaggagtcaaaaaaagtgaacatctctagttctctggaatctgaagatattagtttagaaacaacagttcctacggatgatatttcctcatcagaagagcgagagggcaaagtcagaatcaccaggcagctaattgaacgaaaagaactacttcataatattcagttactaaaaattgagctatcccagaaaactatgatgatcgacaatttgaaagtggattatcttacaaagattgaagaattggaggagaaacttaatgatgcacttcaccagaagcagctactaacattgagattagacaaccaattggcttttcaacagaaagatgccagcaaatatcaagaattaatgaaacaagaaatggaaaccattttgttgagacagaaacaactagaagagacaaatcttcagctaagagaaaaagctggagatgttcgtcgaaacctgcgtgactttgagttgacagaagagcaatatattaaattaaaagcttttcctgaagatcagctttctattcctgaatatgtatctgttcgcttctatgagctagtgaatccattaagaaaggaaatctgtgaactacaagtgaaaaagaatatcctagcagaagaattaagtacaaacaaaaaccaactgaagcagctgacagagacatatgaggaagatcgaaaaaactactctgaagttcaaattagatgtcaacgtttggccttagaattagcagacacaaaacagttaattcagcaaggtgactaccgtcaagagaactatgataaagtcaagagtgaacgtgatgcacttgaacaggaagtaattgagcttaggagaaaacatgaaatacttgaagcctctcacatgattcaaacaaaagaacgaagtgaattatcaaaagagagaccattataaaacagaatatgaaaataaactacatgatgaactagaacaaatcagattgaaaaccaaccaagaaattgatcaacttcgaaatgcctctagggaaatgtatgaacgagaaaacagaaatctccgagaagcaagggataatgctgtggctgaaaaggaacgagcagtgatggctgaaaaggatgctttagaaaaacacgatcagctcttagacaggtacagagaactacaacttagtacagaaagcaaagtaacagaatttctccatcaaagtaaattaaaatcttttgaaagtgagcgtgttcaacttctgcaagaggaaacagcaagaaatctcacacagtgtcaattggaatgtgaaaaatatcagaaaaaattggaggttttaaccaaagaattttatagtctccaagcctcttctgaaaaacgcattactgaacttcaagcacagaactcagagcatcaagcaaggctagacatttatgagaaactggaaaaagagcttgatgaaataataatgcaaactgcagaaattgaaaatgaagatgaggctgaaagggttcttttttcctacggctatggtgctaatgttcccacaacagccaaaagacgactaaagcaaagtgttcacttggcaagaagagtgcttcaattagaaaaacaaaactcgctgattttaaaagatctggaacatcgaaaggaccaagtaacacagctttcacaagagcttgacagagccaattcgctattaaaccagactcaacagccttacaggtatctcattgaatcagtgcgtcagagagattctaagattgattcactgacggaatctattgcacaacttgagaaagatgtcaggaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatggaagggcgaattctgcagatatccatcacactggcggccgctcgagcatgcatctagagggcccaattcgccctatagtgagtcgtattacaaatcacggccgtcag

SEQ ID NO: 22 - DNA, 1880 bp, Homo sapiens
ccgagctcggatccactagtaacggccgccagtgtgctggaattcgcccttctgccttgaaatcccttgttgagggcctacaaccttgtgcttccgactggagacgcctttggtccctcggtgtctgcactggctgctggtcaaggcttcagtgtggagtaattgacactttcgagaatattaaaatcaaattagagaagaaaactgatccataataataaaaatgtctcgaaaaatttcaaaggagtcaaaaaaagtgaacatctctagttctctggaatctgaagatattagtttagaaacaacagttcctacggatgatatttcctcatcagaagagcgagagggcaaagtcagaatcaccaggcagctaattgaacgaaaagaactacttcataatattcagttactaaaaattgagctatcccagaaaactatgatgatcgacaatttgaaagtggattatcttacaaagattgaagaattggaggagaaacttaatgatgcacttcaccagaagcagctactaacattgagattagacaaccaattggcttttcaacagaaagatgccagcaaatatcaagaattaatgaaacaagaaatggaaaccattttgttgagacagaaacaactagaagagacaaatcttcagctaagagaaaaagctggagatgttcgtcgaaacctgcgtgactttgagttgacagaagagcaatatattaaattaaaagcttttcctgaagatcagctttctattcctgaatatgtatctgttcgcttctatgagctagtgaatccattaagaaaggaaatctgtgaactacaagtgaaaaagaatatcctagcagaagaattaagtacaaacaaaaaccaactgaagcagctgacagagacatatgaggaagatcgaaaaaactactctgaagttcaaattagatgtcaacgtttggccttagaattagcagacacaaaacagttaattcagcaaggtgactaccgtcaagagaactatgataaagtcaagagtgaacgtgatgcacttgaacaggaagtaattgagcttaggagaaaacatgaaatacttgaagcctctcacatgattcaaacaaaagaacgaagtgaattatcaaaagagagaccattataaaacagaatatgaaaataaactacatgatgaactagaacaaatcagattgaaaaccaaccaagaaattgatcaacttcgaaatgcctctagggaaatgtatgaacgagaaaacagaaatctccgagaagcaagggataatgctgtggctgaaaaggaacgagcagtgatggctgaaaaggatgctttagaaaaacacgatcagctcttagacaggttttaaccaaagaattttatagtctccaagcctcttctgaaaaacgcattactgaacttcaagcacagaactcagagcatcaagcaaggctagacatttatgagaaactggaaaaagagcttgatgaaataataatgcaaactgcagaaagaattggcagcaatgaaacagattctcgttaagatgcatagtaaacattctgagaacagcttacttctcactaaaacagaaccaaaacatgtgacagaaaatcagaaatcaaagactttgaatgtgcctaaagagcatgaagacaatatatttacacctaaaccaacactctttactaaaaaagaagcacctgagtggtctaagaaacaaaagatgaagacctagtgttttggatgggaagcacctgtagaccattatatactcctgaagttctttttctgatggaagggcgaattctgcagatatccatcacactggcggccgctcgagcatgcatctagagggcccaattcgccctatagtgag

SEQ ID NO: 23 - protein, 157 aa, Mus musculus
MSRKIAKEPKKVNISSSLESEDISLETTIHTDDVSSSEEREGKVKITRQLIERKEILHNIQLLKIELSQKNMMIDNLKMDYLTKEFAAMKQIIINMCSKHSENNLFLTKMESKSVTENQAKTLNMPREHEENIFIPKPTLFTKKEAQEWPKSQKMKT

SEQ ID NO: 24 - DNA, 741 bp, Mus musculus
gtgaattgtaatacgactcactatagggcgaattgggccctctagatgcatgctcgagcggccgccagtgtgatggatatctgcagaattcgcccttatgtctcgcaaaattgccaaggaaccgaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcagaagatgaagacctagtgtttggtgttgaagaaagctggagcctaacccagccaggccctcggctctccattggaacaggcctgtgttatcatgtactcctgaagtgcaatgtctgttggaagggcgaattccagcacactggcggccgttactagtggatccgagctcggtaccaagcttggcgt

SEQ ID NO: 25 - protein, 256 aa, Mus musculus
MSRKIAKEPKKVNISSSLESEDISLETTIHTDDVSSSEEREGKVKITRQLIERKEILHNIQLLKIELSQKNMMIDNLKMDYLTKIEELEEKLNDALHQKQLLTLRLDNQLTIQQKDAKKYQELMKQEMETILLRQRDAKIDSLMKSTAQLEKDVSNLNKEKSALLQTKNQMALDLEQLLSHREEFAAMKQIIINMCSKHSENNLFLTKMESKSVTENQAKTLNMPREHEENIFIPKPTLFTKKEAQEWPKSQKMKT

SEQ ID NO: 26 - DNA, 875 bp, Mus musculus
atgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagattgaggagctagaggaaaaacttaatgacgcccttcaccagaagcagctgctaactttgcgattagacaatcagttgactattcaacagaaagatgccaaaaaatatcaagaactaatgaaacaagaaatggaaaccattttattgcgacagagagatgccaagattgactcactgatgaagtctacagctcaacttgagaaagatgtcagcaacttaaataaagagaagtcagccctgctgcagacgaagaaccagatggcactggatctggagcagctcctcagtcaccgcgaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcagaagatgaagacctagtgtttggtgttgaagaaagctggagcctaacccagccaggccctcggctctccattgggacaggcctgtgttatcatgtactcctgaagtgcaatgtctgttgg

SEQ ID NO: 27 - protein, 229 aa, Mus musculus
MSRKIAKEPKKVNISSSLESEDISLETTIHTDDVSSSEEREGKVKITRQLIERKEILHNIQLLKIELSQKNMMIDNLKMDYLTKLDRANSLLNQTQQPYRYLIESVRQRDAKIDSLMKSTAQLEKDVSNLNKEKSALLQTKNQMALDLEQLLSHREEFAAMKQIIINMCSKHSENNLFLTKMESKSVTENQAKTLNMPREHEENIFIPKPTLFTKKEAQEWPKSQEMKT

SEQ ID NO: 28 - DNA, 908 bp, Mus musculus
cctgagctgctgactgaagacgccattggttcttccaagagtccgggtgaagctggtgttatccttatatgcggagttactgaccattgagagaagattgattcaaataataaaatgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagcttgacagagccaattcactgttgaatcagactcagcagccctacagatacctcatagagtctgtgcgacagagagatgccaagattgactcactgatgaagtctacagctcaacttgagaaagatgtcagcaacttaaataaagagaagtcagccctgctgcagacgaagaaccagatggcactggatctggagcagctcctcagtcaccgcgaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcaggagatgaagacctagtgtttggtgttgaagaaagctggagcctaacccagccaggccctcggctctccattggaacaggcctgtgttatcatgtactcctgaagtgcaatgtctgttgg

SEQ ID NO: 29 - protein, 512 aa, Mus musculus
MSRKIAKEPKKVNISSSLESEDISLETTIHTDDVSSSEEREGKVKITRQLIERKEILHNIQLLKIELSQKNMMIDNLKMDYLTKIEELEEKLNDALHQKQLLTLRLDNQLTIQQKDAKKYQELMKQEMETILLRQKQLEETNHQLREKAGDVRRNLRDFELTEEQYVKLKSFPEDQLSIPEYVSIRFYELVNPLRKEVCELQVKKSELSEELSTSKGQLKQLTETYEEDRRNNAELLIRCQRLTLELADTKQLVQQGDYRQENYDKVKSERDALEQDVLELRRKHEVLEASHIAQAKERNELSKEVSSLQQTVTLLQKDKDYLNRQNMELSVRCAHEEDRLERLQVQLEDTKKAREEMYEKYVTSRDHYKTEYENKLHDELEQIKLKTNLEIDQLRSASREMYERENRNLREARDNALAEENRAVAAEKDALGKHEQLLDRYRELQLSTESKVSEFLHQSKLKSFESERVQLLQEETARNLTQCQLECEKYQKKLETKKEAQEWPKSQKMKT

SEQ ID NO: 30 - DNA, 1806 bp, Mus musculus
gtgaattgtaatacgactcactatagggcgaattgggccctctagatgcatgctcgagcggccgccagtgtgatggatatctgcagaattcgcccttatgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagattgaggagctagaggaaaaacttaatgacgcccttcaccagaagcagctgctaactttgcgattagacaatcagttgactattcaacagaaagatgccaaaaaatatcaagaactaatgaaacaagaaatggaaaccattttattgcgacagaagcaactggaagaaacaaaccatcagctgagagaaaaggctggagatgttcgccgaaatctgcgagactttgagctgacagaagagcagtatgtgaagctaaaatcttttcccgaagatcaactctctattcctgaatatgtatctattcgcttctatgagctcgtgaacccattaagaaaggaagtctgtgagctacaggtgaagaagagtgaactctctgaagaactgagtacaagtaaaggccaactgaagcagctgacggagacatatgaagaagatcgaagaaacaacgctgaacttctaattcgatgtcaacgtttgaccttagaattagcagacacaaaacagttagttcagcaaggtgattaccgtcaagagaactatgacaaagtgaagagtgaacgcgatgctctggaacaggacgtactcgagcttagaagaaaacacgaagtacttgaagcctctcacatagctcaagctaaggaaaggaatgaattatcaaaggaggtcagcagcctgcagcagacagtcaccctgctgcagaaggataaagactacctcaatcgccaaaacatggaactcagtgtacgctgtgcccatgaggaggatcggctggaaaggctgcaagttcaactggaagacaccaaaaaggctagagaagagatgtatgagaaatatgtcacgtccagagaccattataaaacagaatatgaaaataaactacatgatgaactggaacaaatcaaattgaaaactaatctagaaattgatcagcttcgaagtgcctctagggaaatgtatgaacgagaaaacagaaatctccgtgaagcaagggataatgcactcgctgaagagaaccgagcagtggcagcggaaaaggacgctctgggaaagcatgagcagctcctagacaggtacagagaactccagctcagtacagagagcaaggtatctgagtttctccatcagagcaagttgaagtcctttgaaagtgagcgtgttcaactcctgcaagaggaaactgcaagaaatctcacgcagtgccagttggagtgtgaaaaatatcagaagaaattggagactaaaaaggaagcacaagagtggcccaagagtcagaagatgaagacctagtgtttggtgttgaagaaagctggagcctaacccagccaggccctcggctctccatcggaacaggcctgtgttatcatgtactcctgaagtgcaatgtctgttggaagggcgaattccagcacactggcggccgttactagtggatccgagctcggtaccaagcttggcgt

SEQ ID NO: 31 - protein, 569 aa, Mus musculus
MSRKIAKEPKKVNISSSLESEDISLETTIHTDDVSSSEEREGKVKITRQLIERKEILHNIQLLKIELSQKNMMIDNLKMDYLTKIEELEEKLNDALHQKQLLTLRLDNQLTIQQKDAKKYQELMKQEMETILLRQKQLEETNHQLREKAGDVRRNLRDFELTEEQYVKLKSFPEDQLSIPEYVSIRFYELVNPLRKEVCELQVKKSELSEELSTSKGQLKQLTETYEEDRRNNAELLIRCQRLTLELADTKQLVQQGDYRQENYDKVKSERDALEQDVLELRRKHEVLEASHIAQAKERNELSKEVSSLQQTVTLLQKDKDYLNRQNMELSVRCAHEEDRLERLQVQLEDTKKAREEMYEKYVTSRDHYKTEYENKLHDELEQIKLKTNLEIDQLRSASREMYERENRNLREARDNALAEKNRAVAAEKDALGKHEQLLDRYRELQLSTESKVSEFLHQSKLKSFESERVQLLQEETARNLTQCQLECEKYQKKLEEFAAMKQIIINMCSKHSENNLFLTKMESKSVTENQAKTLNMPREHEENIFIPKPTLFTKKEAQEWPKSQKMKT

SEQ ID NO: 32 - protein, 86 aa, Mus musculus
MNYQRRSAACSRQSPCCRRIKTTSIAKTWNSVYAVPMRRIGWKGCKFNWKTPKRLEKRCMRNMSRPETIIKQNMKINYMMNWNKSN

SEQ ID NO: 33 - DNA, 1963 bp, Mus musculus
cgactcactatagggcgaattgggccctctagatgcatgctcgagcggccgccagtgtgatggatatctgcagaattcgcccttatgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagattgaggagctagaggaaaaacttaatgacgcccttcaccagaagcagctgctaactttgcgattagacaatcagttgactattcaacagaaagatgccaaaaaatatcaagaactaatgaaacaagaaatggaaaccattttattgcgacagaagcaactggaagaaacaaaccatcagctgagagaaaaggctggagatgttcgccgaaatctgcgagactttgagctgacagaagagcagtatgtgaagctaaaatcttttcctgaagatcaactctctattcctgaatatgtatctattcgcttctatgagctcgtgaacccattaagaaaggaagtctgtgagctacaggtgaagaagagtgaactctctgaagaactgagtacaagtaaaggccaactgaagcagctgacggagacatatgaagaagatcgaagaaacaacgctgaacttctaattcgatgtcaacgtttgaccttagaattagcagacacaaaacagttagttcagcaaggtgattaccgtcaagagaactatgacaaagtgaagagtgaacgcgatgctctggaacaggacgtactcgagcttagaagaaaacacgaagtacttgaagcctctcacatagctcaagctaaggaaaggaatgaattatcaaaggaggtcagcagcctgcagcagacagtcaccctgctgcagaaggataaagactacctcaatcgccaaaacatggaactcagtgtacgctgtgcccatgaggaggatcggctggaaaggctgcaagttcaactggaagacaccaaaaaggctagagaagagatgtatgagaaatatgtcacgtccagagaccattataaaacagaatatgaaaataaactacatgatgaactggaacaaatcaaattgaaaactaatctagaaattgatcagcttcgaagtgcctctagggaaatgtatgaacgagaaaacagaaatctccgtgaagcaagggataatgcactcgctgaaaagaaccgagcagtggcagcggaaaaggacgctctgggaaagcatgagcagctcctagacaggtacagagaactccagctcagtacagagagcaaggtatctgagtttctccatcagagcaagttgaagtcctttgaaagtgagcgtgttcaactcctgcaagaggaaactgcaagaaatctcacgcagtgccagttggagtgtgaaaaatatcagaagaaattggaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcagaagatgaagacctagtgtttggtgttgaagaaagctggagcctaacccagccaggccctcggctctccattggaacaggcctgtgttatcatgtactcctgaagtgcaatgtctgttggaagggcgaattccagcacactggcggccgttactagtggatccgagctcggtaccaagcttggcg

SEQ ID NO: 34 - protein, 309 aa, Mus musculus
MYEKYVTSRDHYKTEYENKLHDELEQIKLKTNLEIDQLRSASREMYERENRNLREARDNALAEKNRAVAAEKDALGKHEQLLDRYRELQLSTESKVSEFLHQSKLKSFESERVQLLQEETARNLTQCQLECEKYQKKLEVLTKEFYSLQTSSEKRITELEAQNSEHQARLDIYEKLEKELDEIIMQTAEIENEDEAERILYSYGYGANVPTTAKRRLKQSVHLARRVLQLEKQNSLILKDLEQIIINMCSKHSENNLFLTKMESISVTENQTKTLNMPREHEENIFIPKPTLFTKKEAQEWPKSQKMKT

SEQ ID NO: 35 - DNA, 1169 bp, Mus musculus
ggttgtcttggtgaccgatcctaacggtccggtagccccgagatacctgttgaggggtggcagcctgagctgctgactgaagacaccaaaaaggctagagaagagatgtatgagaaatatgtcacgtccagagaccattataaaacagaatatgaaaataaactacatgatgaactggaacaaatcaaattgaaaactaatctagaaattgatcagcttcgaagtgcctctagggaaatgtatgaacgagaaaacagaaatctccgtgaagcaagggataatgcactcgctgaaaagaaccgagcagtggcagcggaaaaggacgctctgggaaagcatgagcagctcctagacaggtacagagaactccagctcagtacagagagcaaggtatctgagtttctccatcagagcaagttgaagtcctttgaaagtgagcgtgttcaactcctgcaagaggaaactgcaagaaatctcacgcagtgccagttggagtgtgaaaaatatcagaagaaattggaggttttaactaaagaattttatagtctccaaacttcttctgaaaaacgcattaccgaactcgaggcacagaactcagagcatcaggcaaggttagacatttacgagaagttggaaaaggagcttgatgagataataatgcaaacagcagaaattgaaaatgaagatgaggctgaaagaattctttattcctatggttatggtgctaatgttcccacaacagctaaaagacgactaaagcaaagtgtccacttggcaagaagagttcttcagttagaaaaacaaaattcattaattttaaaagacctggagcagatcatcattaatatgtgtagtaaacattctgagaacaacttatttcttacgaaaatggaatcaataagtgtgacagaaaatcaaacaaagactttgaatatgccaagagaacatgaagagaatatatttatacccaagccaacgctctttactaaaaaggaagcacaagagtggcccaagagtcagaagatgaagacctagtgtttggtgttgaagaaagctggagcctaacccagccaggccctcggctctccattggaacaggcctgtgttatcatgtactcctgaagtgcaatgtctgttggggagaaaggctcacttcaatcatgtacttg

SEQ ID NO: 36 - protein, 641 aa, Mus musculus
MSRKIAKEPKKVNISSSLESEDISLETTIHTDDVSSSEEREGKVKITRQLIERKEILHNIQLLKIELSQKNMMIDNLKMDYLTKIEELEEKLNDALHQKQLLTLRLDNQLTIQQKDAKKYQELMKQEMETILLRQKQLEETNHQLREKAGDVRRNLRDFELTEEQYVKLKSFPEDQLSIPEYVSIRFYELVNPLRKEVCELQVKKSELSEELSTSKGQLKQLTETYEEDRRNNAELLIRCQRLTLELADTKQLVQQGDYRQENYDKVKSERDALEQDVLELRRKHEVLEASHIAQAKERNELSKEVSSLQQTVTLLQKDKDYLNRQNMELSVRCAHEEDRLERLQVQLEDTKKAREEMYEKYVTSRDHYKTEYENKLHDELEQIKLKTNLEIDQLRSASREMYERENRNLREARDNALAEKNRAVAAKKDALGKHEQLLDRYRELQLSTESKVSEFLHQSKLKSFESERVQLLQEETARNLTQCQLECEKYQKKLELDRANSLLNQTQQPYRYLIESVRQRDAKIDSLMKSTAQLEEDVSNLNKEKSALLQTKNQMALDLEQLLSHREEFAAMKQIIINMCSKHSENNLFLTKMESKSVTENQAKTLNMPREHEENIFIPKPTLFTKKEAQEWPKSQKMKT

SEQ ID NO: 37 - DNA, 2144 bp, Mus musculus
cctgagctgctgactgaagacgccattggttcttccaagagtccgggtgaagctggtgttatccttatatgcggagttactgaccattgagagaagattgattcaaataataaaatgtctcgcaaaattgccaaggaaccaaaaaaagtaaatatctctagttctctggagtctgaagatattagtttggaaacaaccattcatacagatgatgtctcatcatcagaggagcgagaaggtaaagtcaaaatcaccaggcagttaatcgaaagaaaagagatacttcataatattcagttactgaaaatcgagctatcccagaaaaacatgatgatcgacaacttgaaaatggattatcttacaaagattgaggagctagaggaaaaacttaatgacgcccttcaccagaagcagctgctaactttgcgattagacaatcagttgactattcaacagaaagatgccaaaaaatatcaagaactaatgaaacaagaaatggaaaccattttattgcgacagaagcaactggaagaaacaaaccatcagctgagagaaaaggctggagatgttcgccgaaatctgcgagactttgagctgacagaagagcagtatgtgaagctaaaatcttttcctgaagatcaactctctattcctgaatatgtatctattcgcttctatgagctcgtgaacccattaagaaaggaagtctgtgagctacaggtgaagaagagtgaactctctgaagaactgagtacaagtaaaggccaactgaagcagctgacggagacatatgaagaagatcgaagaaacaacgctgaacttctaattcgatgtcaacgtttgaccttagaattagcagacacaaaacagttagttcagcaaggtgattaccgtcaagagaactatgacaaagtgaagagtgaacgcgatgctctggaacaggacgtactcgagcttagaagaaaacacgaagtacttgaagcctcacacatagctcaagctaaggaaaggaatgaattatcaaaggaggtcagcagcctgcagcagacagtcaccctgctgcagaaggataaagactacctcaatcgccaaaacatggaactcagtgtacgctgtgcccatgaggaggatcggctggaaaggctgcaagttcaactggaagacaccaaaaaggctagagaagagatgtatgagaaatatgtcacgtccagagaccattataaaacagaatatgaaaataaactacatgatgaactggaacaaatcaaattgaaaactaatctagaaattgatcagcttcgaagtgcctctagggaaatgtatgaacgagaaaacagaaatctccgtgaagcaagggataatgcactcgctgaaaagaaccgagcagtggcagcgaaaaaggacgctctgggaaagcatgagcagctcctagacaggtacagagaactccagctcagcacagagagcaaggtatctgagtttctccatcagagcaagttgaagtcctttgaaagtgagcgtgttcaactcctgcaagaggaaactgcaagaaatctcacgcagtgccagttggagtgtgaaaaatatcagaagaaattggagcttgacagagccaattcactgttgaatcagactcagcagccctacagatacctcatagagtctgtgcgacagagagatgccaagattgactcactgatgaagtctacagctcaacttgaggaagatgtcagcaacttaaataaagagaagtcagccctgctgcagacgaagaaccagatggcactggatctggagcagctcctcagtcaccgcgaggaatttgcagctatgaagcagatcatcattaatatgtgtagtaaacattctgagaacaacttatttcttacgaaaatggaatcaaaaagtgtgacagaaaatcaagcaaagactttgaatatgccaagagaacatgaagagaatatatttatacccaagccaacgctctttactaaaaaggaagcacaagagtggcccaagagtcagaagatgaagacctagtgtttggtgttgaagaaggctggagcctaacccagccaggccctcggctctccattggaacaggcctgtgttatcatgtactcctgaagtgcaatgtctgttgg